(19) United States
(12) Patent Application Publication
(10) Pub. No.: US 2005/0164388 A1
(43) Pub. Date: Jul. 28, 2005

(54) Method of isolating epithelial cells, method of preconditioning cells, and methods of preparing bioartificial skin and dermis with the epithelial cells and preconditioned cells

(76) Inventors: Young-Sook Son, Seoul (KR); Hyun-Sook Park, Seoul (KR); Chun-Ho Kim, Seoul (KR); Hyun-Ju Kang, Seoul (KR); Chang-Hwan Kim, Kyungki-do (KR); Youn-Young Kim, Seoul (KR); Young-Ju Choi, Seoul (KR); Su-Hyun Lee, Seoul (KR); Yong-Jae Gin, Seoul (KR)

Correspondence Address: CANTOR COLBURN, LLP, 55 GRIFFIN ROAD SOUTH, BLOOMFIELD, CT, 06002, US

(21) Appl. No.: 10/258,987

(22) PCT Filed: Nov. 6, 2001
(86) PCT No.: PCT/KR01/01873

(30) Foreign Application Priority Data
Feb. 7, 2001 (KR) 2001/5934
Aug. 8, 2001 (KR) 2001/47723

(51) Int. Cl. C12N 5/08; A61F 2/10

(52) U.S. Cl. 435/381; 623/15.12; 435/383; 623/915

(57) ABSTRACT

A method of isolating epithelial cells from a human skin tissue or internal organ tissue using trypsin and ethylene-diamine tetraacetic acid (EDTA) simultaneously with the application of magnetic stirring, a method of preconditioning isolated biological cells by the application of physical stimulus, i.e., strain, are provided. Epithelial cells can be isolated by the method with increased yield, colony forming efficiency (CFE), and colony size. Also, the increased percentage of stem cells in isolated cells is advantageous in therapeutic tissue implantation by autologous or allogeneic transplantation. In skin cells preconditioned by the application of strain, cell division is facilitated, and the secretion of extracellular matrix components and growth factors and the activity of matrix metalloproteinases (MMPs) are improved. When preconditioned cells are implanted by autologous or allogeneic transplantation to heal a damaged tissue, the improved cell adhesion, mobility, and viability provides a biological adjustment effect against a variety of stresses or physical stimuli which the cells would undergo after implantation, with improved capability of integration into host tissue, thereby markedly improving the probability of success in skin grafting.

### TECHNICAL FIELD

The present invention relates to a method of isolating epithelial cells and a method of preconditioning cells, and more particularly, to a method of isolating epithelial cells from skin or internal organs using trypsin and ethylenediamine tetraacetic acid (EDTA) simultaneously with magnetic stirring, and a method of in vitro preconditioning isolated skin cells with the application of physical stimuli during cell culture.

### BACKGROUND ART

Human skin tissue is roughly divided into three parts: the epidermis on top, the dermis underneath, and the subcutaneous tissue. The epidermis consists of epithelial cells stratified from the basement membrane between the epidermis and the dermis, and melanocytes and Langerhans Cells. The dermis consists of fibroblasts and extracellular matrix secreted by the fibroblasts.

Skin epithelial cells have different cell ages and degrees of differentiation for each cell layer. This is because stem cells in the basal layer downregulate the number of integrin receptors as cell division progresses and migrate to upper cell layers. The upper layers of epithelial cells are much more differentiated than lower layers, and finally the uppermost (outer) layer loses nuclei and forms a keratin layer through concretion of keratin remaining therein. The major function of skin epithelial cells is to protect the body from the exterior environment by forming keratin. Therefore, skin epithelial cells are also called “keratinocytes”. The keratin layer is periodically separated from the epidermis and supplemented by new cells generated through cell division in the basal cell layer membrane such that the epidermis keeps a constant number of cells. The basal cell layer includes stem cells and transit amplifying cells divided from the stem cells. It is difficult to identify these two types of cells from each other. However, some recent reports demonstrated that, stem cells, unlike transit amplifying cells, showed predominant β₁-integrin expression and high adhesion to the basement membrane, which is considered to be related with β₁-integrin expression. Stem cells with predominant β₁-integrin expression are known to be located on the rete ridge of the basement membrane, occupying about 4-10% of the basal cell layer. When cultured on culture dishes, stem cells typically showed a high colony forming efficiency and a slow cell division rate (Bickenbach and Chism, 1998, ECR 244:184-195; Jones and Watt et al., 1993, Cell 73:713-7124).

Stem cells are present in all epithelial cells including skin epidermis. It is known that stem cells of the cornea are present in the basal layer of the limbus of the cornea. In esophagus and vagina among internal human organ, stem cells are present in the basal cell layer. Mucosal epithelial cells of the stomach and small and large intestines having glandular structures are formed as a single cell layer and have stem cells deep within their glandular structures. In conclusion, stem cells for any epithelial cells are present deep within and unexposed in their structures, at sites referred to as “stem cell niche”. Therefore, stem cells are expected not to be easily separated, compared to other cells.

Skin tissue or internal organ may be partially damaged by burn, traumatic injury, ulcer, etc. To heal a wound tissue or for plastic surgery, grafting of epithelial cells (keratinocytes), which are cultured after being separated from the patient's or another person's skin tissue or internal organ, onto damaged skin or internal organ has been widely used. To this end, a need exists for effective techniques of separating epithelial cells. In addition, the percentage of stem cells in separated cells should be high enough to ensure high cell expansion potential in the culture environment and successful implantation.

A conventional cell isolation method developed by Rheinwald and Green in 1975 (hereinafter, “Green's method”) has been widely used for separation and culture of human primary epithelial cells. According to this method, epidermal cells are separated using trypsin and EDTA or using only trypsin with or without gentle shaking. Green's method provides a sufficiently high cell yield for research purpose cell isolation, but not enough for cell isolation for tissue engineering-based industrial use.

More recently, a 2-step enzyme treatment method (hereinafter, “thermolysin method” or “dispase method”), in which epidermis-dermis separation using an enzyme is followed by enzymatic epithelial cell (keratinocytes) separation from the epidermis, has been introduced. In this method, skin tissue is pre-treated with thermolysin (Germain et al., 1993, Burns 19:99-104) or dispase (Simon and Green, 1985, Cell 40:677-683) to separate the epidermis and dermis from each other, followed by separation of the epidermis into individual epidermal cells with treatment of trypsin/EDTA. Thermolysin is known for specifically to break the epidermis-dermis junction of skin with reactivity between bullous pemphigoid antigen and laminin, without destroying desmosomes. Isolation of the epidermis from the dermis with thermolysin or dispase is advantageous in that contamination of fibroblast is reduced. Disadvantageously, however, inactivation of thermolysin or dispase cannot be controlled in the 2-step enzyme treatment method. These two enzymes are known to retain its function in an enzyme-substrate complex for a while after epidermis separation so that undesirable damage of cells may occur after the epidermis separation. This probable cell damage was proven from the results of a 2-step enzyme treatment method-by the present inventors as shown in FIG. 4, whereby epithelial cells isolated by the 2-step enzyme treatment method showed a low colony forming efficiency (CFE).

Epithelial cells (keratinocytes) separated with conventional methods such as Green's method or the 2-step enzyme treatment method are reported as showing limited rounds of cell propagation in primary cell culture and keep only a portion of the cells grafted onto a patient's skin after autologous transplantation. This is emerging as a significant problem in epithelial cell grafting. A low percentage of stem cells in separated epithelial cells would be one reason for the problem. In consideration of the complex rete ridge structure and strong binding capability of stem cells to the basement membrane, conventional methods are ineffective in isolating basal cells, particularly stem cells, from the basement membrane. This is evident in FIG. 1 where a considerable percentage of basal cells remains in a tissue sample after cell isolation from skin.

The present inventors assumed that addition of trypsin and EDTA simultaneously with vigorous physical agitation would be efficient in separating basal cells. The present inventors also expected that the yield of stem cells be considerably increased. In other words, the present inventors have improved the separation of epithelial cells by applying magnetic stirring in addition to the treatment with trypsin and EDTA (hereinafter, “magnetic stirring method”). In order to prove the efficiency of the magnetic stirring method in separating epithelial cells including enriched stem cells, the present inventors have separated epithelial cells from skin tissue and compared the magnetic stirring method with the existing cell isolation methods, such as Green's method, thermolysin method, and dispase method, for cell yield, CFE, and colony size (cell numbers per colony) of the separated epithelial cells. As a result, the magnetic stirring method according to the present invention showed greater cell yield, CFE, and colony size than the three existing cell isolation methods.

Culture of isolated cells as well as cell isolation itself described above are crucial to ensuring high cell expansion potential in the culture environment and successive grafting.

A variety of primary human cell cultures are used in skin grafting to treat skin damage. However, poor cell viability and low intake ratio of primary skin cell cultures into a host tissue makes it difficult successful skin grafting (Burke al., 1981, Ann Surg 194:413-428, 1981). Cell necrosis is considered to occur since the implanted cells fail to adapt to various stresses and physical stimuli in the tissue. Therefore, there is a need for a new culturing technique improving the intake ratio into host tissue with enhanced cell viability.

Research reports based on cartilage or tibial tissue supported a close relationship between physical stimuli and cell differentiation (Tagile and Aspenberg, 1999, J. Orthop Res 17:2004; Aspenberg et al., 2000, Acta Orthop Scand 71:558-62). For this reason, during primary cartilage culture, compression is applied to induce cell differentiation.

There are some reports on the effect of strain as a physical stimulus applied in vivo to tendon or cardiac fibroblast, on the mitogenesis or extracellular matrix synthesis.

In the case where only strain is applied to avian tendon fibroblasts or rat cardiac fibroblasts, there is no significant effect on the mitogenesis and pro-collagen synthesis. Fibroblast mitogenesis is slightly stimulated when platelet derived growth factor (PDFG-BB) and insulin-like growth factor (IGF-I) are incorporated along with the application of strain. Pro-collagen synthesis is facilitated about 2-4 times more when fetal bovine serum (FBS) and transforming growth factor (TGF-β) are supplemented (Banes et al, 1995, J. Biomechanics 28:1505-1513; Butt and Bishop, 1997, J. Mol. Cell Cardiol 29:1141-1151).

Main components of extracellular matrix of the dermis which are closely associated with satisfactory skin grafting include fibronectin, elastin, glycosaminoglycan (GAG) as well as collagen. In particular, fibronectin is known to be present in both tissue and blood and to be synthesized in vascular endothelial cells, fibroblasts, myoblasts, epithelial cells, nerve cells, etc. Fibronectin, a dimer composed of two polypeptides linked together (220 KD), contributes cell attachment to other cells or collagen or cell migration. Most of all, fibronectin as an extracellular matrix component that supports the initial stage of wound healing is essential for adhesion and migration of fibroblasts, vascular endothelial cells, and keratinocytes (Yamada and Clark, 1996, Provisional Matrix, from the Molecular and Cellular Biology of Wound Repair: 51-93).

Major wound healing components secreted by dermal fibroblasts include matrix metalloproteinase (MMP)-2 and MMP-9. MMP-2 and MMP-9 support the remodeling of extracellular matrix in wound healing progress, mitogenesis, and angiogenesis and affects the migration of epithelial cells and vascular endothelial cells (Yu et al., 1998, 72-kDa Gelatinase (Gelatinase A): Structure, Activation, Regulation, and Substrate Specificity, from Matrix Metalloproteinases: 85-113). In particular, MMP-9 is generated within a few hours after injury and shows increased expression in keratocytes migrating for re-epithelialization. Thus, MMP-9 is considered to be significant in migrating keratocytes and in the early stage of wound healing (Vu and Werb, 1998, Galatinase B: Structure, Regulation, and Function, from Matrix Matalloproteinases: 115-147; Parks et al., 1998, Matrix Metalloproteinase, from Matrix Metalloproteinases: 85-113).

As described above, the present invention has been launched based upon the fact that poor adaptation of implant cells to stress and physical stimuli in the human tissue hinders successful skin grafting. Also, the effects of the present invention have been verified through experiments for identifying the indices of skin grafting and data analysis thereof.

### DISCLOSURE OF THE INVENTION

To overcome the above problems of conventional cell isolation methods, it is a first object of the present invention to provide a new method of isolating epithelial cells with increased cell yield, CFE, and colony size (proportion of stem cells).

It is a second object of the present invention to provide a method of preconditioning dermal fibroblasts, keranocytes, or vascular endothelial cells in vitro by the application of strain for successful skin grafting.

It is a third object of the present invention to provide methods of preparing a bioartificial skin or bioartificial dermis with good implant effect by using epithelial cells separated by one of the above methods or cells preconditioned by the other method.

It is a fourth object of the present invention to provide an effective method of curing skin tissue or internal organ damaged by burns, traumatic injury, or ulcer by implantation of isolated epithelial cells, preconditioned cells, or a bioartificial skin or bioartificial dermis, which are obtained by one of the methods described above.

To achieve the first object of the present invention, there is provided a method of isolating epithelial cells by treating skin tissue or internal organ with trypsin and EDTA simultaneously with magnetic stirring. In the present invention as a modification of a conventional method, Green's method, a single cell suspension is obtained by the enzymatic reaction of trypsin and EDTA simultaneously with the application of physical force by vigorous magnetic stirring. The skin tissue or internal organ may be obtained from any animal skin or organ. It is preferable that the skin tissue is obtained from the foreskin, axilla, hip, abdomen, breast, scalp, cornea, pubes, or marsupium, and the internal organ tissue is obtained from the oral cavity mucosa, esophagus mucosa, gastric mucosa, intestinal mucosa, nasal cavity, gorge, bronchus, kidney, urethra, uterus mucosa, bladder, or vagina.

In the present invention, treatment with trypsin and EDTA may be performed by a well-known method, Green's method (Rheinwald and Green, 1975). It is preferable that trypsin is added in an amount of 0.025%-0.25%, and EDTA is added in an amount of 0.005-0.02%. If the amounts of trypsin and EDTA are less than the above ranges, easy cell isolation is not ensured. If the amounts of trypsin and EDTA exceed the above ranges, the number of colonies is markedly reduced due to damage of cells.

It is preferable that magnetic stirring is carried out at 60-700 rpm, more preferably 150-500 rpm, for 10 minutes to 4 hours. If the rate of magnetic stirring is not greater than 60 rpm, cells are not easily separated. If the rate of magnetic stirring is greater than 700 rpm, the number of colonies is reduced due to damage of cells. The magnetic stirring in the cell isolation method according to the present invention facilitates cell isolation by weakening the binding force of basal cells to the basement membrane.

To achieve the second object of the present invention, there is provided a method of preconditioning isolated skin cells in vitro in cultures with the application of physical stimulus, i.e., strain. According to this method, a physical stimulus is additionally applied to skin cells before implantation based upon a conventional primary cell culture method to precondition the skin cells against various physical stresses that the skin cells would undergo after being implanted into a body tissue.

In the preconditioning method, physical stimulus is generated by vacuum and adjusted in a computerized, pressure-oriented system, such as a Bio-Stretch system or Flexercell Strain Plus™ system, or its equivalents. These systems can apply strain to inoculated cells and support medium by elongating a culture plate with a rubber bottom by using vacuum pressure. It is preferable that strain is pulsed or is constantly applied at a frequency of 0.1-3.0 Hz at 0.01-40% maximum strain (elongation). If the maximum strain is smaller than the above range, physical stimulus is not applied to cells. If the maximum strain is greater than the above range, undesirably cells are damaged or cell adhesion is weakened.

The in vitro cell preconditioning method according to the present invention now will be described in greater detail.

To easily attach cells on the rubber bottom of a 6-well plate type I-P collagen (Cell Matrix, Gelatin Corp.) or type I-A collagen (Cell Matrix, Gelatin Corp) is coated on the 6-well plate. Fibronectin and/or glyoseaminoglycan (GAG) may be additionally coated on the collagen coated 6-well plate to improve cell adhesion and propagation. Cells are inoculated on the plate coated with collagen or other extracelluar matrix components and cultured in appropriate media until confluency reaches 80-90%. The culture medium is changed once every two days and switched to a serum-free medium for cell preconditioning. During cell preconditioning, strain is pulsed or is constantly applied at a frequency of 0.1-3.0 Hz at 0.01-40% maximum strain, with or without the addition of suitable growth factors or serum. It is preferable that cells subjected to preconditioning are fibroblasts, vascular endothelial cells (VECs), or keratinocytes. Preferably, strain is applied at 0.5-15% maximum strain for dermal fibroblasts, 10-30% maximum strain for VECS, and 0.1-30% maximum strain for keratinocytes.

To achieve the third object of the present invention, there is provided a method of preparing a bioartificial skin by inoculating the epithelial cells isolated by the magnetic stirring method in an artificial dermal construct or de-epidermized dermis (DED), exclusively or together with fibroblasts at the same time or sequentially.

In the present invention, any commercially available artificial dermal constructs can be used, for example, neutralized chitosan sponge, a mixed sponge of neutralized chitosan and collagen (BAS™, MTT) which are admitted by FDA or under request for FDA's authentication, Integra® (Integra LifeSciences), Alloderm (LifeCell), Terudermis (Terumo Co.), or Beschitin W (Unitika Ltd.). DED used for the preparation of the bioartificial skin may be obtained from a human corpse or animals.

Also, to achieve the third object of the present invention, there is provided a method of preparing a bioartificial skin by inoculating epithelial cells along with melanocytes, hair follicle cells, or dermal sheath in an artificial dermal construct.

In addition, the third object of the present invention is achieved by a method of preparing a bioartificial dermis by inoculating fibroblasts in an artificial dermal construct or DED, and a method of implanting the bioartificial dermis in a body tissue for wound healing, tissue expansion, or plastic surgery.

The third object of the present invention is also achieved by a method of preparing a bioartificial dermis by inoculating VECs exclusively or along with fibroblasts in an artificial dermal construct.

In the method of preparing a bioartificial skin or bioartificial dermis described above, epithelial cells and/or fibroblasts isolated and cultured by the methods according to the present invention are loaded at a density of 1×10⁴-1×10⁶ cells/cm² (scaffold). In the present invention, dynamic seeding of cells in a dermal construct using a shaker is followed by dynamic culturing. Alternatively, static seeding and static culturing in which cells are inoculated in a dermal construct and cultured without the application of flow, can be used.

To achieve the fourth object of the present invention, there is provided a method of curing a damaged skin or internal organ by implanting epithelial cells isolated by the method according to the present invention in a damaged skin tissue or internal organ, exclusively or along with dermal fibroblasts.

The fourth object of the present invention is also achieved by a method of curing a damaged tissue or internal organ by implanting a bioartificial skin or bioartificial dermis in a damaged skin tissue or internal organ, the bioartificial skin or bioartificial dermis prepared by implanting epithelial cells and dermal fibroblasts isolated by the method according to the present invention in an artificial dermal construct.

In the present invention, isolated cells can be implanted by autologus or allogeneic transplantation according to the method (Wang et al., 2000, JID 114:674-680) known well in the field.

In the present invention, the damaged skin tissue to be repaired may include not only a tissue site damaged by burns, traumatic injury, or ulcer, but also a tissue site that needs skin plastic surgery or external tissue expansion. Also, the internal organic tissue may include the oral cavity mucosa, esophagus mucosa, gastric mucosa, intestinal mucosa, nasal cavity, gorge, bronchus, kidney, urethra, uterus mucosa, bladder, and vagina.

The bioartificial skin or bioartificial dermis prepared by the method according to the present invention can be used as a model for a variety of clinical, research, and testing purposes. For example, the bioartificial skin or bioartificial dermis prepared by the method according to the present invention can be used as a model for testing the toxicity or efficacy of cosmetic source materials, a model for pharmaceutical skin permeability or pharmaceutical efficacy or toxicity test, a model for testing the efficacy of trichogen, a model for wound healing research, a model for research on cell migration or penetration, invasion, or progress of tumor cells, a model for angiogenesis research or for testing the efficacy of angiogenesis stimulator or inhibitor, or a model for research cell differentiation, interaction of epithelial cells, basal cells, and VECs, or the function of protein or gene.

The present inventors compared the cell isolation method by magnetic stirring according to the present invention with conventional methods, Green's method, Thermolysin method (Germain et al., 1993, Burns 19199-104), and Dispase method (Simon and Green, 1985, Cell 40:677-683), for cell yield, CFE and colony size. As a result, relative cell yields by the magnetic stirring method was 6.3 fold with respect to Green's method, 2.2 fold with respect to Thermolysin method, and 4.9 fold with respect to Dispase method, as shown in FIGS. 2 and 3. Relative CFEs by the magnetic stirring method was 1.2 fold with respect to Green's method, 4.2 fold with respect to Thermolysin method, and 1.4 fold with respect to Dispase method, as shown in FIG. 4. In addition, the number of colony forming cells (stem cells) per foreskin sample in the magnetic stirring method, which is a product of cell yield by CFE, was 7.2 fold with respect to Green's method, 9.2 fold with respect to Thermolysin method, and 6.9 fold with respect to Dispase method, as shown in FIG. 5.

For the cell isolation method by magnetic stirring according to the present invention, the level of β₁ integrin expression in the surface of the cell was skewed to the right (increase), as shown in FIG. 6. This means that the percentage of integrin-bright cells as a stem cell maker, in which integrin is predominantly expressed, is increased by magnetic stirring. In contrast, the percentage of involucrin-positive cells (involucrin as a terminal differentiation marker), was low in the magnetic stirring method, compared to the other isolation methods. In conclusion, the cell isolation method by magnetic stirring according to the present invention inhibits terminal differentiation with improved cell yield and CFE. Therefore, the cell isolation method by magnetic stirring according to the present invention is considered to be the most suitable cell isolation method for cell expansion with retarded cell differentiation and aging effect. Due to the increase in the percentage of stem cells, the cell isolation method by magnetic stirring according to the present invention is suitable for skin grafting.

The third object of the present invention is also achieved by a method of preparing a bioartificial dermis with in vitro preconditioned cells. In the preparation is of a bioartificial dermis, the fibroblasts and/or VECs preconditioned by the in vitro cell preconditioning method described above are inoculated in an artificial or native dermal construct by a dynamic and/or static method at a density of 1×10³-1×10⁷ cells/cm³.

In an alternative method of forming a bioartificial dermis, fibroblasts and/or VECs are inoculated in an artificial or native dermal construct by the same method above at a density of 110³-110⁷ cells/cm³, and subjected to preconditioning as in the in vitro cell preconditioning method, with the application of physical stimulus.

Alternatively, in the preparation of a bioartificial dermis, collagen solution or fibrin solution can be used as a dermal construct.

Alternatively, in the preparation of a bioartificial dermis according to the present invention, the fibroblasts and/or VECs preconditioned by the in vitro cell preconditioning method described above are mixed in a collagen solution or fibrin solution at a density of 1×10³-1×10⁷ cells/cm³, and gelated.

Alternatively, in the preparation of a bioartificial dermis according to the present invention, the fibroblasts and/or VECs preconditioned by the in vitro cell preconditioning method described above are mixed in a collagen solution or fibrin solution at a density of 1×10³-1×10⁷ cells/cm³, gelated, and subjected to physical stimulus as in the in vitro cell preconditioning method.

Alternatively, in the preparation of a bioartificial dermis according to the present invention, fibroblasts and/or VECs which are not preconditioned are mixed in a collagen solution or fibrin solution at a density of 1×10³-1×10⁷ cells/cm³, gelated, and subjected to physical stimulus as in the in vitro cell preconditioning method described above.

Preferably, the physical stimulus applied in the preparation of a bioartificial dermis may be strain applied under the same conditions as the in vitro cell preconditioning method described above. The conditions for preparing a bioartificial dermis can be varied according to the shape or type of artificial dermal construct used therefor or the purpose of clinical tests performed with the prepared artificial dermis.

In the preparation of a bioartificial dermis according to the present invention, the dermal construct used therefore may include a native dermal construct such as DED, collagen solution, fibrin solution, gelated collagen, and gelated fibrin, and any commercially available artificial dermal construct. Suitable artificial dermal constructs may include neutralized chitosan sponge, a mixed sponge of neutralized chitosan and collagen (BAS™, MTT), Integra® (Integra LifeSciences), Alloderm (LifeCell), Terudermis (Terumo Co.), and Beschitin W (Unitika Ltd.).

In the preparation of a bioartificial dermis, fibronectin and/or glycoseaminoglycan (GAG) may be added to a dermal construct used.

The third object of the present invention is also achieved by a method of preparing a bioartificial skin, in which epithelial cells preconditioned by the in vitro cell preconditioning method described above are inoculated in a dermal construct at a density of 1×10³-1×10⁷ cells/cm³ in a static manner.

Alternatively, in the preparation of a bioartificial skin according to the present invention, epithelial cells which are not preconditioned are inoculated at a density of 1×10³-1×10⁷ cells/cm³ in a static manner, and physical stimulus as in the in vitro cell preconditioning method described above is applied thereto. The physical stimulus applied in the preparation of a bioartificial skin may be strain applied under the same condition as in the in vitro cell preconditioning method described above.

In the preparation of a bioartificial skin according to the present invention, the dermal construct used therefore may include native and artificial dermal constructs, the bioartificial dermis prepared by the method described above, and a boiartificial dermal construct by other methods. Suitable artificial dermal constructs may include neutralized chitosan sponge, a mixed sponge of neutralized chitosan and collagen (BAS™, MTT), Integra® (Integra LifeSciences), Alloderm (LifeCell), Terudermis (Terumo Co.), and Beschitin W (Unitika Ltd.).

The epithelial cells used in the preparation of a bioartificial skin may include keratinocytes and melanocytes separately or both keratinocytes and melanocytes. In the preparation of a bioartificial skin, it is preferable that either melanocytes, hair follicle cells, or dermal sheath, or all of the previous are inoculated.

To achieve the fourth object of the present invention, there is also provided a method of healing a damaged tissue by implanting the bioartificial dermis or bioartificial skin prepared by the method described above. There is also provided a method of healing a damaged tissue by directly implanting the keratinocytes, fibroblasts, or VECs preconditioned by the in vitro cell preconditioning method described above, in an implant site of damaged skin tissue or internal organic tissue. The implantation of a bioartificial dermis or bioartificial skin, and the inoculation of keratinocytes, fibroblasts, or VECs are performed by the methods known in the arts.

The present inventors have verified the effect of in vitro preconditioning on a variety of dermal cells, such as fibroblasts, VECs, and keratinocytes, in the following examples.

### BRIEF DESCRIPTION OF THE DRAWINGS

FIG. 1 shows photographs of adult human foreskins stained with hematoxylin and eosin (H&E) after cell isolation by a variety of cell isolation methods.

FIG. 2 illustrates a variety of methods of isolating epithelial cells;

FIG. 3 shows the cell yield for the different cell isolation methods;

FIG. 4 shows the colony forming efficiency (CFE) for the different cell isolation methods;

FIG. 5 are graphs comparatively showing the CFE and the number of colony forming cells per foreskin sample for the different cell isolation methods;

FIG. 6 shows the levels of β1 Integrin expression by flow cytometry in keratinocytes isolated by the different cell isolation methods;

FIG. 7 are photographs of immunostaining for the expression of involucrin of primary keratinocytes isolated by the different cell isolation methods;

FIG. 8 are photographs of immunofluorescent staining of primary keratinocytes isolated by the magnetic stirring method for the expression of involucrin, pan-cytokeratin, and α₂ integrin;

FIG. 9 illustrates the implantation procedure of keratinocytes, which were isolated by a magnetic stirring method according to the present invention, together with fibroblasts into a nude mouse;

FIG. 10 shows a photograph immunohistochemistry of human skin for the expression of human pan-cytokeratin, human vimentin, human collagen IV and human laminin-5;

FIG. 11 shows a H&E staining and immunohistochemistry for pan-cytokeratin of stratified epidermal keratinocytes on DED;

FIG. 12 shows scanning electromicroscopic (SEM) photographs of fibroblasts inoculated in a bioartificial skin construct (BAS™) and incubated for 14 days;

FIG. 13 shows SEM photographs (a) of fibroblasts inoculated in DED and incubated for 21 days and a photograph (b) of the same stained with H&E;

FIG. 14 shows photographs of H&E staining of fibroblasts inoculated in artificial dermal constructs (Integra® and Terumdermis) and incubated for 14 days;

FIG. 15 shows the implantation of an artificial dermal construct (Integra® or Terumdermis) in which fibroblasts were inoculated in DED and incubated for 14 days, into the back of a nude mouse;

FIG. 16 are photographs showing the level of elevation of the implant sites of mice 28 days after implantation of an artificial dermal construct or a boiartificial dermal construct (Integra® or Terumdermis) and photographs of H&E staining for the same tissue;

FIG. 17 shows the variations in height of the artificial dermal constructs and the bioartificial dermal constructs of FIG. 16;

FIG. 18 shows the relative cell density of dermal fibroblasts in a bioartificial skin construct (BAS™) between static and dynamic methods, which is a measure of cell growth and division rates;

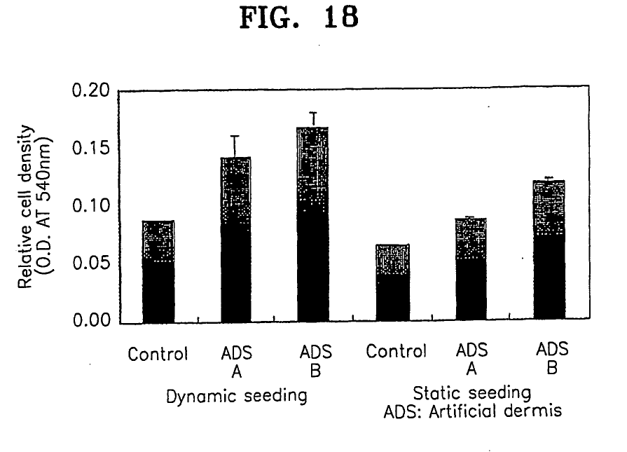

FIG. 19 shows phase contrast microscopic photographs showing increases in the number of cells after newborn human fibroblasts are preconditioned with the application of strain using a FX-4000T™ in Example 8;

FIG. 20 shows the result of a Western blot assay for variations in Cyclin-D1 expression after newborn human fibroblasts are preconditioned with the application of strain using a FX-4000T™ in Example 8, and the comparison to a growth factor treated group;

FIG. 21 shows the result of an immunoprecipitation assay for the levels of fibronectin and collagen secretion in cell culture media after newborn and adult dermal fibroblasts are preconditioned with the application of strain using a FX-4000T™ in Example 8, and the comparison to a growth factor treated group;

FIG. 22 shows the result of an immunoprecipitation assay for the level of fibronectin secretion in cell culture media after keratinocytes are preconditioned with the application of strain using a FX4000T™ in Example 10;

FIG. 23 shows the result of immunostaining for variation in the expression of collagen IV after human umbilical vein endothelial cells (HUVECs) are preconditioned with the application of strain using a FX-4000T™ in Example 9;

FIG. 24 shows photographs of immunofluorescent staining for filbronectin and photographs of cell nuclei stained with DAPI after adult fibroblasts are preconditioned with the application of strain using a FX-4000T™ in Example 8, inoculated on a coverslip, and cultured for 4 days;

FIG. 25 shows photographs of immunofluorescent staining for α-smooth muscle actin and photographs of cell nuclei stained with DAPI after newborn and adult fibroblasts are preconditioned with the application of strain using a FX-4000T™ in Example 8, inoculated on a coverslip, and cultured for 4 days;

FIG. 26 shows the result of zymography for the activity of matrix metalloproteinases (MMPs) in cell culture media after keratinocytes (a) and dermal fibroblasts (b) are preconditioned with the application of strain using a FX4000T™;

FIG. 27 shows the result of flow cytometry for the levels of HLA-ABC (histocompatibility antigen) expression carried out after each sub-culturing in Example 11 with adult fibroblasts, in which (b) is a table and a graph obtained based upon the data of (a); and

FIG. 28 shows the result of quantification of vascular endothelial growth factor (VEGF) by ELISA after fibroblasts and vascular endothelial cells (VECs) and keratinocytes are preconditioned with the application of strain using a FX-4000T™, with and without the addition of VEGF.

### BEST MODE FOR CARRYING OUT THE INVENTION

The present invention will be described in greater detail by means of the following examples. The following examples are for illustrative purposes and are not intended to limit the scope of the invention.

### EXAMPLE 1

### Cell Isolation and Culture

Primary keratinocytes were isolated from adult human foreskins obtained by circumcision. The adult human foreskins were placed in an epidermal minimal medium (hereinafter, E-medium) containing 1% penicillin, streptomycin, and 250 ng/ml Fungizone (Cat. No. 15240-062, Gibco) at 4° C. before cell isolation. Primary keratinocytes were isolated not later than 24 hours from circumcision.

The foreskin sample was washed at least 8 times in a phosphate buffered saline (PBS) solution containing 5% penicillin/streptomycin. Subcutaneous tissue was mostly removed from the dermis of the foreskin sample with a pair of sterile surgical scissors, and the remaining portion was cut into tissue fragments not larger than 1-2 mm².

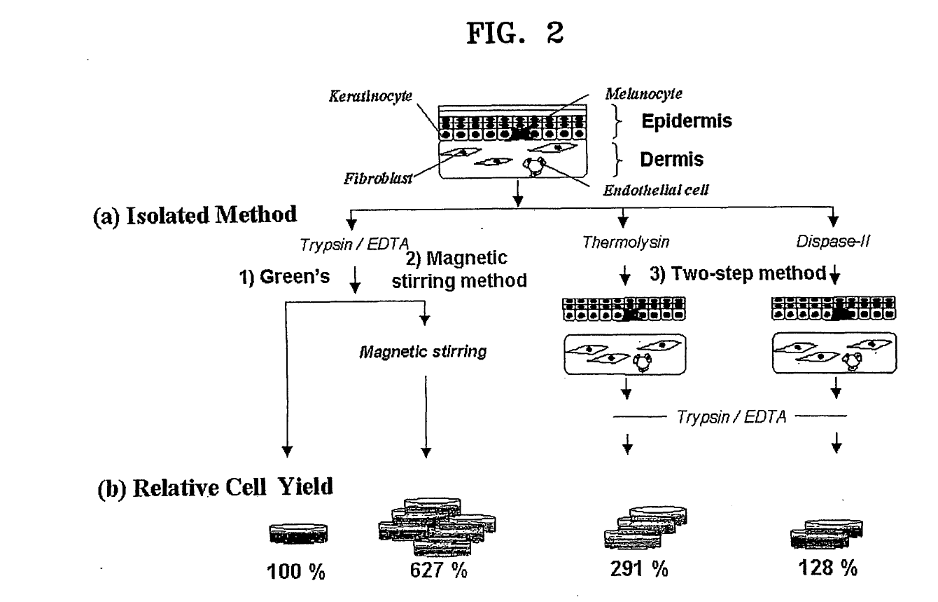

Cell isolation was carried out by four methods, (i) magnetic stirring method according to the present invention, and conventional methods including (ii) Green's method, (iii) thermolysin method, and (iv) dispase method, based upon the procedures described in references, and the results of the four methods were compared (refer to FIG. 2).

(i) Magnetic Stirring Method

Tissue fragments were placed in 10 ml of 0.00125% trypsin and 0.01% ethylenediamine tetraacetic acid (EDTA) for 30 minutes with magnetic stirring at 100 rpm to isolate cells. The isolated cells were washed in a 10 ml E-medium containing 20% fetal bovine serum to inactivate trypsin and were recovered by centrifugation. The cell pellets were resuspended in Keratinocyte Growth Medium (KGM) (Cat No. CC-3111, Clonetics BioWhittaker, Walkersville) and then inoculated in a culture plate at a density of 5×10³/cm². This experiment was carried out three times.

(ii) Green's Method

Tissue fragments were incubated for 30 minutes at 37° C. in 10 ml of 0.025% trypsin solution with single voltexing every 5 minutes to isolate cells. The isolated cells were washed in a 10 ml E-medium containing 20% fetal bovine serum to inactivate trypsin and were recovered by centrifugation. The cell pellets were resuspended in KGM (Cat No. CC-3111, Clonetics BioWhittaker, Walkersville) and then inoculated in a culture plate at a density of 5×10³/cm². This experiment was carried out three times.

(iii) Thermolysin Method

Tissue fragments were treated in a thermolysin solution (250 μg/ml, Cat No. P1512, Sigma-Aldrich Korea) at 37° C. for 4 hours. After epidermis separation and washing, the resultant cell suspension was further incubated for 30 minutes at 37° C. in 10 ml of 0.05% trypsin and EDTA with shaking. The isolated cells were washed in a 10 ml E-medium containing 20% fetal bovine serum to inactivate trypsin and were recovered by centrifugation. The cell pellets were resuspended in KGM (Cat No. CC-3111, Clonetics BioWhittaker, Walkersville) and then inoculated in a culture plate at a density of 5×10³/cm².

(iv) Dispase Method

Tissue fragments were treated in a dispase II solution (2.4 U/ml, Cat No. 165859, Roche, Mannheim) at 37° C. for 4 hours. After epidermis separation and washing, the resultant cell suspension was further incubated for 30 minutes at 37° C. in 10 ml of 0.05% trypsin and EDTA with shaking. The isolated cells were washed in a 10 ml E-medium containing 20% fetal bovine serum to inactivate trypsin and were recovered by centrifugation. The cell pellets were resuspended in KGM (Cat No. CC-3111, Clonetics BioWhittaker, Walkersville) and then inoculated in a culture plate at a density of 5×10³/cm².

Cells isolated according to the four different methods were examined to determine cell yield (refer to Effect 1 of the present invention) or cell purity (refer to Effect 2) after having been plated on respective coverslips at the densities described above, or examined to identify integrin expression (refer to Effect 4) or involucrin expression (refer to Effect 5). After a 2-week incubation, cells inoculated on the culture plates were examined to determine CFE (refer to Effect 3) or the percentage of β₁-integrin (acting as a stem cell marker) bright cells by flow cytometry as in Example 2. Alternatively, whether or not the cultured cells differentiated into skin cells was determined by direct implantation of the cultured cells into nude mice as in Example 4 (refer to Effect 6) or whether or not the cultured cells differentiated into skin cells by inoculation in de-epidermized dermis (DED) as in Example 5 (refer to Effect 7).

### EXAMPLE 2

### Fluorescence Activated Cell Sorting (FACS)

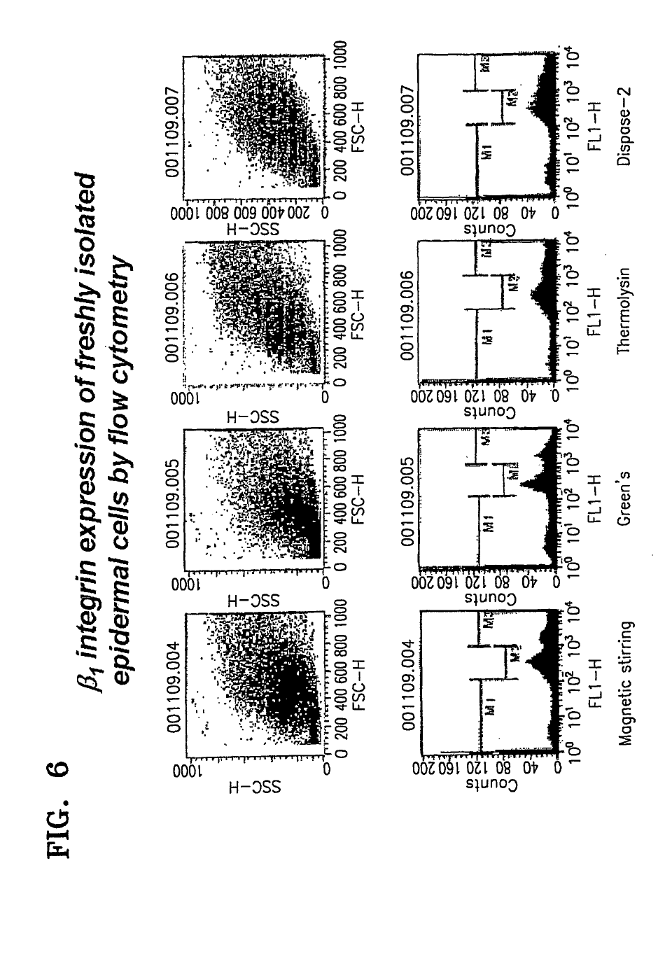

Levels of β₁-integrin expression in cells isolated in Example 1 according to the four methods were compared by FACS to measure the percentage of β₁-integrin bright cells in the isolated cells, which could be predominantly expressed with β₁-integrin known as a stem cell marker. The cells isolated by the respective four methods were incubated along with β₁-integrin antibodies (Chemicon) and followed with fluorescein isothiocyanate (FITC)-conjugated goat anti-mouse antibodies on ice for 45 minutes. The cells were washed in phosphate buffered saline (PBS) containing 5% bovine serum albumin (BSA). At the end of staining, cells were resuspended in a medium at a density of 1×10⁶ cells/ml and sorted using a FACStar^{Plus} (Beckton Dickinson). At least 10,000 cells were analyzed by flow cytometry in each experiment. The results of each experiment was calibrated using fluorescent native antibodies and isotype control antibodies (refer to Effect 4 and FIG. 6).

### EXAMPLE 3

### Immunostaining

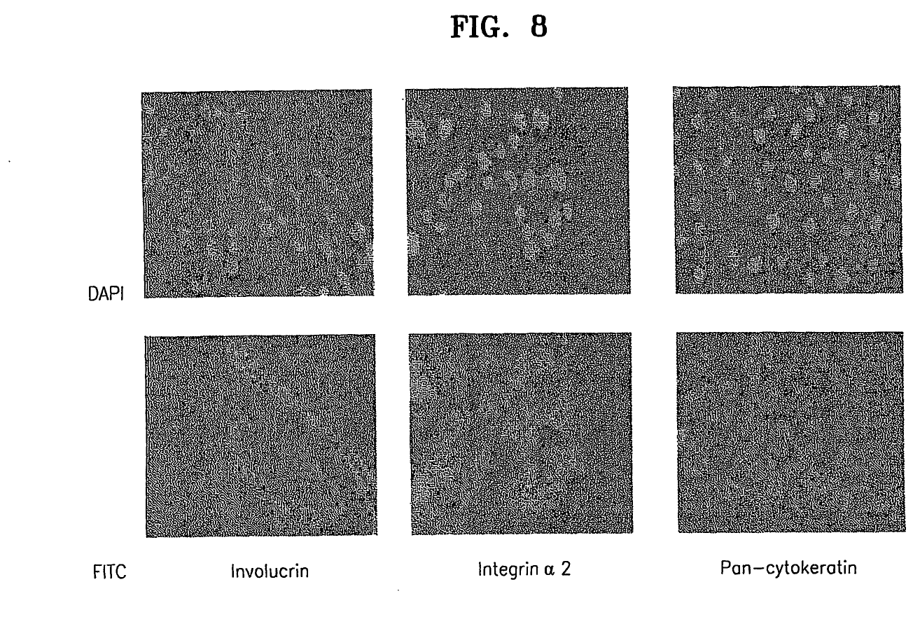

Keratinocytes isolated in Example 1 were cultured on coverslips and fixed for 10 minutes at 4° C. in a 1:1 mixture of ethanol and methanol. To identify whether the isolated and cultured cells exclusively consisted of keratinocytes, the fixed cells were stained with pan-cytokeratin antibodies acting as an epithelial cell marker (refer to FIG. 8 and Effect 2). In addition, the fixed cells were stained with α₂ integrin antibodies (chemicon) to determine whether the isolated and cultured cells showed basal cell characteristics (refer to FIG. 8 and Effect 4), and with involucrin antibodies to determine the number of differentiating cells (refer to FIG. 8 and. Effect 5). The β₁ integrin and α₂ integrin antibodies used were mouse monoclonal antibodies, and the pan-cytokeratin (Novocastra) and Involucrin (Biomedical Technologies, a keratonicyte differentiation indicator) antibodies used were rabbit polyclonial antibodies. Cell incubation in the presence of primary antibodies was followed by staining using a standard ABC kit (Vector Laboratories).

### EXAMPLE 4

### Differentiation of Keratinocyte Implant into Skin of Mouse

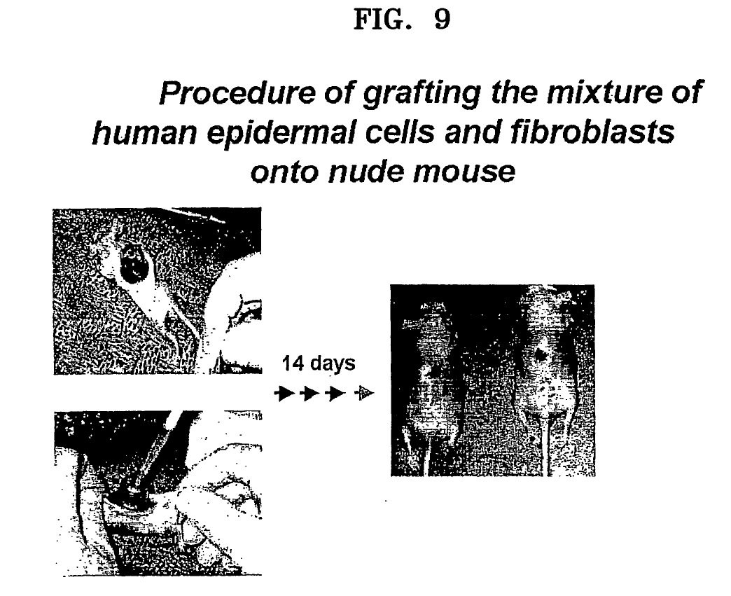

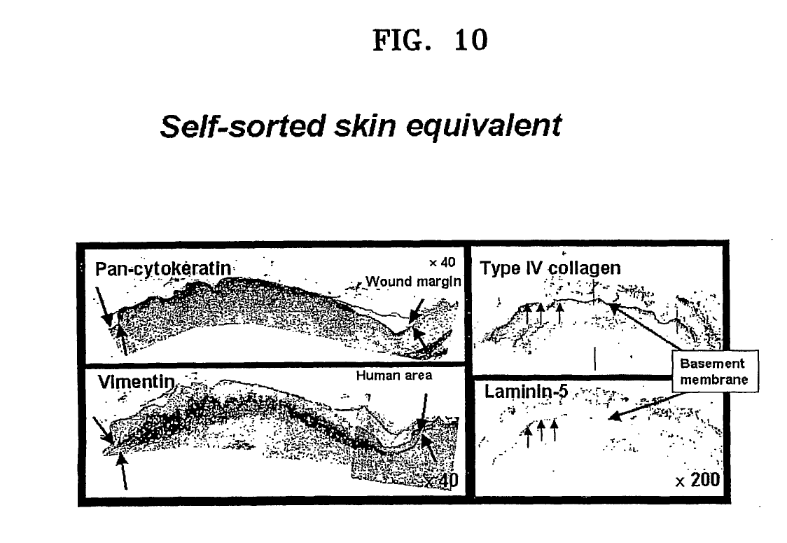

To investigate whether isolated keratinocytes could be successfully differentiated in vivo into skin tissue, the isolated human keratinocytes were implanted into a nude mouse (refer to FIG. 9 and Effect 6). A full thickness incision of 1-cm diameter was made on the back of the mouse, and a plastic chamber was placed into the incision. A cell suspension in KGM containing keratinocytes cultured in Example 1 and dermal fibroblasts were inoculated at a density of 5×10⁵ cells/cm² and 1×10⁵ cells/cm², respectively, into the plastic chamber placed in the mouse The plastic chamber was removed from the body of the mouse after 1 week to induce epidermis differentiation. A portion of the regenerated skin tissue was removed, fixed in 3.7% formalin/PBS, and stained with appropriate reagents including hematoxylin and eosin to verify proliferation of the implanted cells into skin tissue (refer to FIG. 10).

### EXAMPLE 5

### Keratinocyte Differentiation on DED into Skin Epidermis

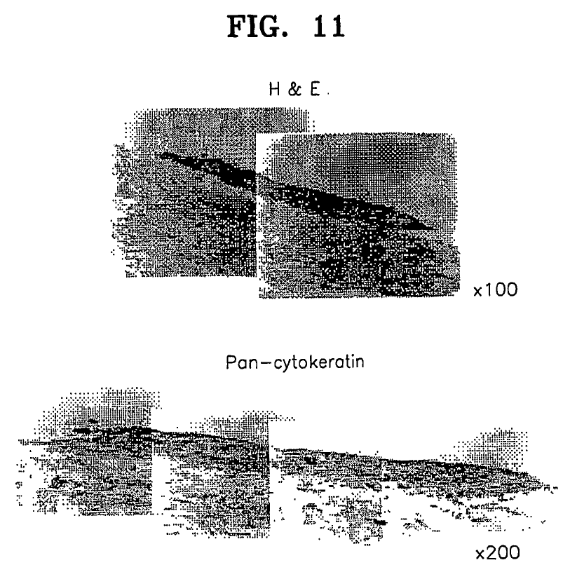

To investigate whether isolated keratinocytes and fibroblasts could be successfully differentiated in vitro into skin tissue, the isolated keratinocytes and fibroblasts were inoculated in a de-epidermized dermis (DED) from a human corpse and incubated for 3 weeks (refer to FIG. 11 and Effect 7). In particular, fibroblasts were inoculated into the bottom dermal reticulus at a density of 1×10⁵ cells/cm², and then 1 day later keratinocytes were inoculated onto the top dermal papillarus at a density of 5×10⁵ cells/cm². The resultant DED was cultured for 1 week, in the submerged state and incubated on an air-liquid interface for 2 weeks. A portion of the resultant culture was removed, fixed in 3.7% formalin/PBS, and stained with appropriate reagents including hematoxylin and eosin to verify proliferation of the cell cultures into skin tissue.

### EXAMPLES 6 and 7

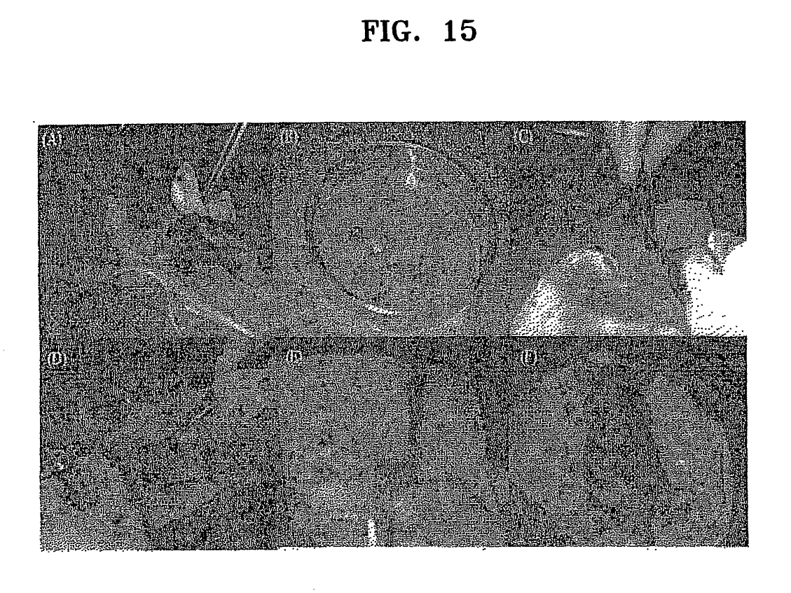

Bioartificial skin may be prepared with or without fibroblasts. In the present embodiments, bioartificial skin with fibroblasts was constructed in vivo and in vitro. For in vivo preparation, fibroblasts were isolated and cultured and subjected to in vivo inoculation to form dermis (refer to FIGS. 9 and 10 and Effect 6). For in vitro preparation, dermal fibroblasts were inoculated into an artificial dermis to obtain a bioartificial dermis (refer to FIGS. 11, 12, 13, and 14 and Effect 7), followed by in vivo transplantation (refer to FIG. 15 and Effect 8).

### EXAMPLE 6

### Inoculation of Fibroblasts in Artificial Dermal Construct

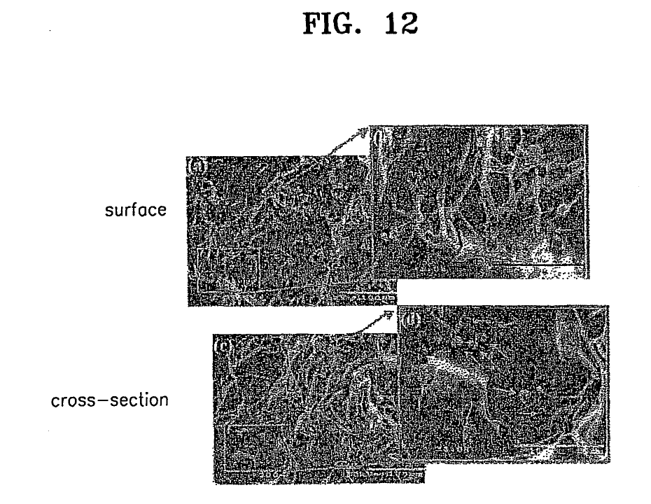

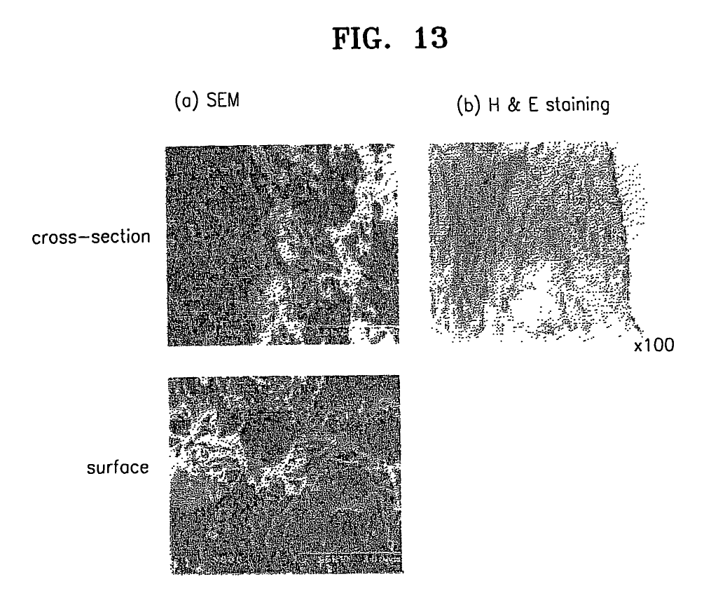

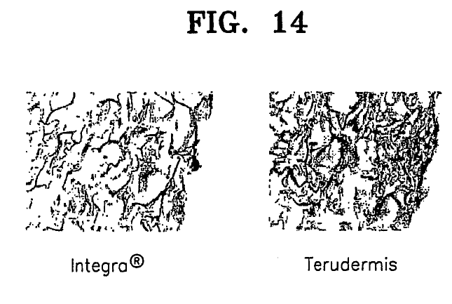

The dermis was separated from adult human foreskins by the methods of Example 1, i.e., with a pair of sterile scissors (Magnetic Stirring Method and Green's method), or by treatment with thermolysin (Thermolysin Method) or dispase (Dispase Method). The separated dermis was soaked in 10 ml of 0.07% collagenase solution and incubated at 37° C. for 2 hours. Then fibroblasts were isolated from the culture by pipetting. The isolated fibroblasts were cultured in a F-medium (Dulbecco's minimal essential medium (DMEM):F-12=3:1) containing 10% fetal bovine serum (FBS) and 1% penicillin/streptomycin) and immediately inoculated in an artificial dermal construct. Alternatively, the cell could be frozen in a preservative solution containing 50% DMEM, 40% FBS, and 10% dimethyl sulfoxide (DMSO) and thawed before inoculating in an artificial dermal construct. Artificial dermal constructs were punctured into a diameter of 8-10 mm in a sterile hood and placed in 24-well culture plates each having a diameter of 10 mm. To prepare bioartificial dermis of 8-mm diameter, 1×10⁵ viable cells (determined using trypan blue exclusion) were diluted in a minimum volume of the DMEM culture solution and inoculated in the punctured dermal constructs uniformly for stable binding with the same. The dermal constructs used were Bioartificial skin (BAS™, refer to FIG. 12 and Effect 8), Integra® (refer to FIG. 14, Effect 8), Alloderm (LifeCell), Terudermis (refer to FIG. 14 and Effect 8) (Terumo Co., Japan), Beschitin W (Unitika Ltd., Japan), and de-epidermized dermis (DED) (refer to FIG. 13 and Effect 8). The dermal constructs inoculated with the fibrobrast culture were maintained at 37° C. under 5% CO₂ in air for 3-5 hours, and 50 μl of the DMEM culture solution was added to each well of the culture plates and 1 ml of the culture solution was added to each after 24 hours. The artificial dermal constructs were incubated under the same conditions for 3-4 weeks to obtain bioartificial dermises with changes of medium performed three times weekly.

### EXAMPLE 7

### Mouse Implantation of Bioartificial Dermis and Artificial Dermal Construct

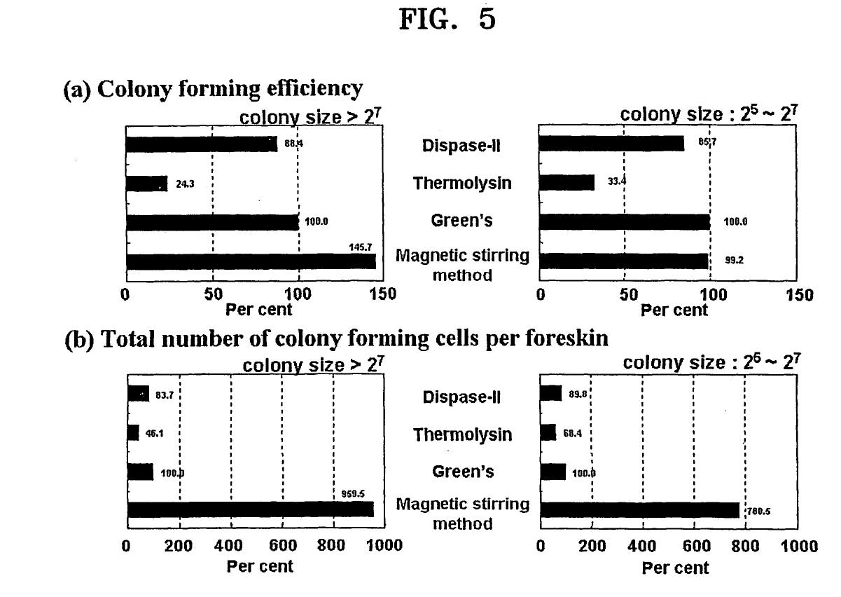

The effect of tissue expansion was verified by implanting the bioartificial dermis prepared by the method of Example 6 and pure artificial dermal constructs into mice (Refer to FIGS. 5 and 6 and Effect 9). The bioartificial dermis used was prepared by inoculating dermal fibroblasts in the artificial dermal constructs, Integra® and Terudermis, and the pure artificial dermal constructs were Integra® and Terudermis. Nude mice were bred in a sterile chamber. A 1-cm wide incision was made in the back of the mice. The bioartificial dermis and the artificial dermal constructs, each having a diameter of 8 mm, were implanted on the fascia of the respective mice using forceps, sealed with sutures, and covered with sterile gauze. Water containing antibiotics, ampicillin and streptomycin, was supplied to the mice to prevent infection. The height of the implant sites of the experimental mice was measured everyday, and sacrificed after 28 days. A tissue sample containing intact skin and the implant site was separated from the mice for histological analysis. The tissue sample was fixed in 3.7% formalin/PBS, paraffin embedded, sectioned, and stained with hematoxylin and eosin.

### EXAMPLE 8

### Preconditioning of Dermal Fibroblasts

Newborn human foreskins from circumcision or adult skin tissue were washed 10 times or more in PBS containing penicillin and streptomycin immediately after circumcision and cut into 2-mm tissue fractions. The tissue fractions were treated overnight with a 2.4 U/mL dispase at 4° C. to isolate keratinocytes, followed by treatment with 0.35% collagenase at 37° C. for 2 hours to isolate single dermal fibroblasts. The isolated single dermal fibroblasts were cultured in a F-medium (DMEM:F-12=3:1) containing 10% FBS or 10% newborn bovine serum and subjected to sub-culturing whenever the cells reached about 80% confluency. Fibroblasts from the fourth passage were inoculated at a density of 3×10⁴ cells/well, incubated in a F-medium for 8 days with changes of medium performed once every 2 days, and subjected to preconditioning. For preconditioning, the dermal fibroblasts were switched to 2 mL of a serum-free medium without addition of any growth factor or with addition of 50 ng/mL platelet-derived growth factor (PDBF)-BB, 10 ng/mL insulin-like growth factor (IGF-I), or 50 ng/mL PDBF-BB and 10 ng/mL IGF-I. Strain was applied to the dermal fibroblasts for preconditioning with a FX-4000T™ for 2 days at 37° C. at a frequency of 1.0 Hz at 10% maximum strain. A control sample was cultured under the same conditions without application of strain.

After preconditioning of the dermal fibroblasts, the dermal fibroblasts were separated by trypsinization, inoculated on a collagen IV-coated coverslip having a diameter of 13 mm, and cultured in a F-medium. Intercellular fibronectin was immunofluorescently stained, and cell nuclei were stained with DAPI to determine whether cell preconditioning effect was lasted.

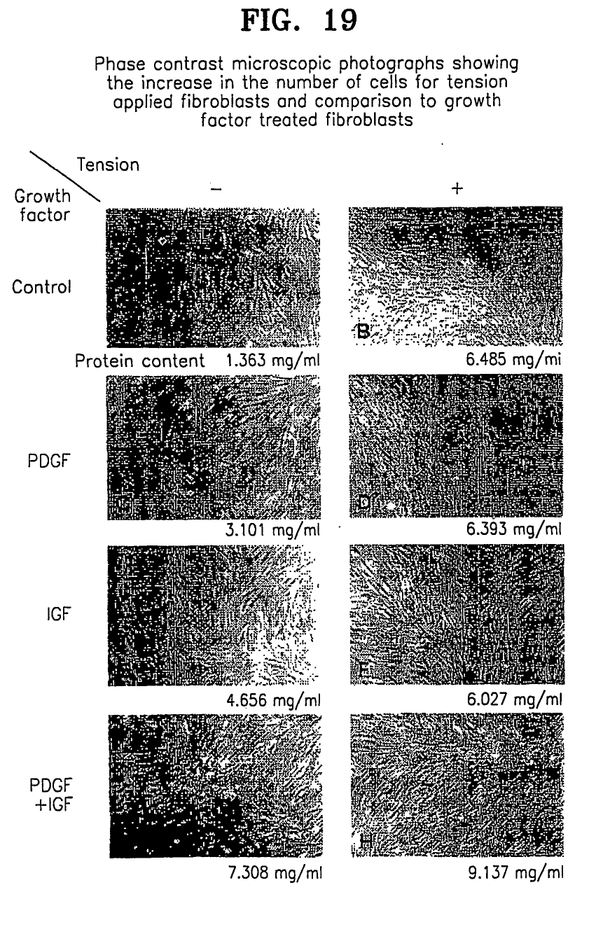

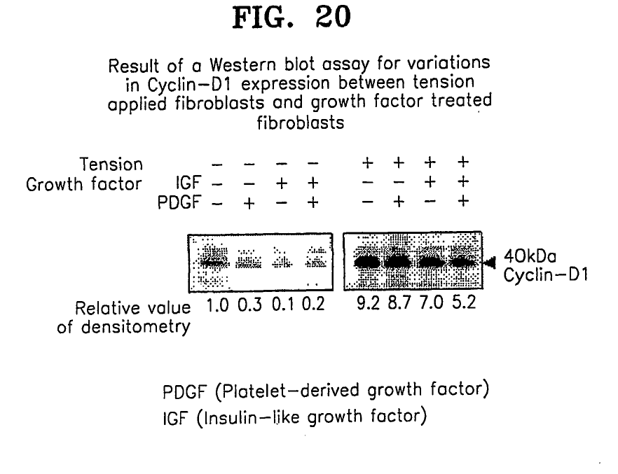

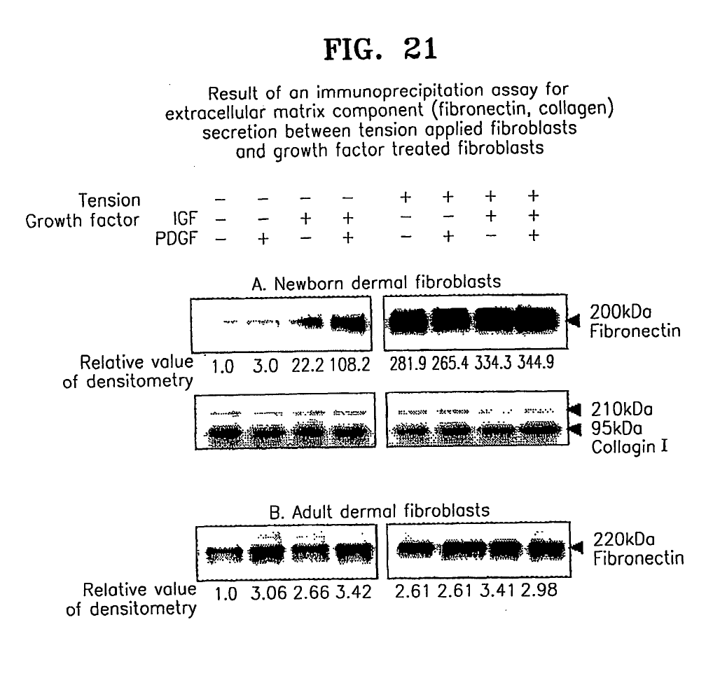

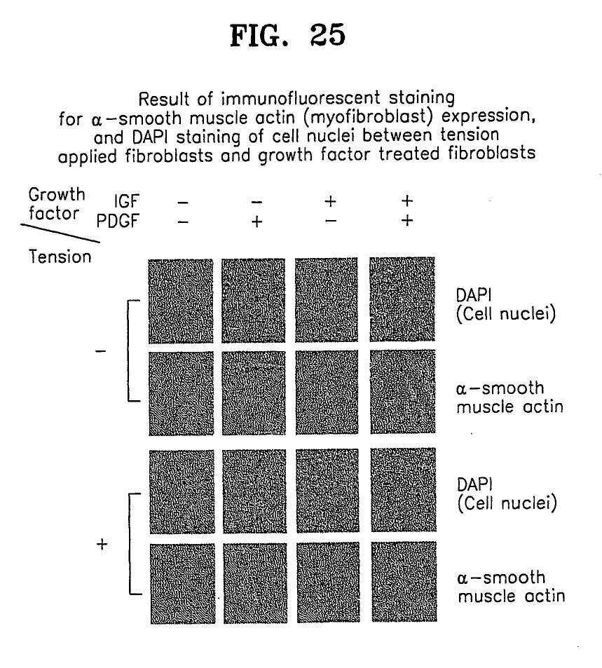

An increase in total protein content of the dermal fibroblasts and variations in cell number by the cell preconditioning were verified (refer to Effect 10 and FIG. 19). Increased cyclin-D1 expression associated with mitogenesis was measured by Western blot analysis (refer to Effect 11 and FIG. 20), and an increase in extracellular matrix component (fibronectin) secretion in cell media was measured by immunoprecipitation assay (refer to Effect 12 and FIG. 21). It was ascertained by immunofluorescent staining that dermal fibroblasts did not convert to myofibroblasts (refer to Effect 14 and FIG. 25). Increased activity of matrix metalloproteinases (MMPs) in culture media was detected by zymography (refer to Effect 15). Lasting cell preconditioning effects were verified by immunofluorescent staining 4 and 7 days after inoculation on coverlips.

### EXAMPLE 9

### Preconditioning of Vascular Endothelial Cells (VECs)

Human umbilical vein endothelial cells (HUVECs) from the fourth passage were inoculated at a density of 2×10⁵ cells/well and left a day for cell adhesion. The HUVECs were cultured in an endothelial growth medium (EGM)-MV (Clonetics Inc.) for 2 days with the application of strain using a FX-4000T™ at a frequency of 1.0 Hz at 15% maximum strain. A control sample was cultured under the same conditions without application of strain.

After preconditioning, increases in the level of collagen IV as an extracellular matrix component in the HUVECs were measured by immunostaining (refer to Effect 12). Increases in vascular endothelial growth factor (VEGF) secretion in culture media were verified by enzyme-linked immunosorbent assay (ELISA) (refer to Effect 17).

### EXAMPLE 10

### Preconditioning of Skin Keratinocytes

Skin keratinocytes from the third passage were inoculated at a density of 510⁵ cells/well and cultured in a KGM. Following changes of medium, the skin keratinocytes were cultured for 2 days with the application of strain using a FX4000T™ at a frequency of 0.5 Hz at 20% maximum strain. A control sample was cultured under the same conditions without application of strain.

After preconditioning, increases in fibronectin secretion in the skin keratinocytes were measured by an immunoprecititation assay (refer to Effect 12). Increased activity of MMPs in culture media were verified by zymography (refer to Effect 15).

### EXAMPLE 11

### Applicability of Allogeneic Fibroblasts for Wound Healing Therapy; Measurement of HLA-ABC Expression Reduction Caused by Fibroblast Sub-Culturing

Human adult fibroblasts were isolated from foreskin samples, reacted with MACS anti-fibroblast microbeads (Miltenyi Biotec.) for 1 hour at room temperature, and subjected to column separation to obtain pure fibroblasts. The isolated fibroblasts were inoculated at a density of 1×10⁵ cells/100-mm culture dish and subjected to sub-culturing whenever the cells reached 80-90% confluency. F-media were used with changes of medium performed once every 2 days. Fibroblasts from the first passage were subjected to FACS for the expression levels of HLA-ABC (Dako) and HLA-DR (Neomarkers). As a result, HLA-DR was not expressed. For this reason, HLA-DR expression was not analyzed for the following passages. For the FACS analysis, the isolated fibroblasts were treated with trypsin, washed in a FACS reagent, and reacted with HLA-ABC antibodies (Dako) and HLA-DR antibodies (Neomarkers) and then with FITC-conjugated secondary antibodies. The cell concentration was adjusted at 5×10⁵-1×10⁶ cells/mL for FACS analysis (refer to Effect 16).

### EXAMPLE 12

### Total Intracellular Protein Content Analysis

For quantification of total intracellular protein, cell plates (BioFlex) were washed in PBS and subjected to cytolysis at 4° C. for 20 minutes in a cell lysis buffer (20 mM Tris-HCl at pH 7.4, 150 mM NaCl, 1 mM Na₂EDTA, 1 mM EGTA, 1% TritonX-100, 2.5 mM sodium pyrophosphate, 1 mM Na₃VO₄, 1 mM β-glycerophosphate, and 1 μg/mL leupeptin) with addition of 2 mM phenylmethyl sulfonylfluoride (PMSF) acting as a protease inhibitor. The cell lysates were scraped with a cell scraper and centrifuged at 4° C. at 12,000 rpm for 20 minutes. The supernatant from the centrifugation was collected for intercellular protein analysis performed using bicinchoninic acid (BCA). 10 μl of the supernatant was added to 2 mL of a 49:1 solution mixture of BCA and 4% CuSO₄ and reacted with the solution mixture at 37° C. for 30 minutes. Following this, the absorbance of the sample was measured spectrophotometrically at 562 nm. The intercellular protein content was determined by comparison to a bovine serum albumin (BSA) standard curve.

### EXAMPLE 13

### Immunoprecipitation

Following cell preconditioning with a FX-4000T™, cell culture media were preserved for cell secretion analysis. Proteins of interest in cell culture media were quantified based upon cell number per unit area of the cell culture plates.

Concanavalin A-sepharose 4B was added to a predetermined amount of a cell culture medium and reacted in a rotator at 4° C. for more than 2 hours. The resultant cells were washed three times in a cell lysis buffer (1% Tx-100, 50 mM Tris-Cl at pH 7.4, 150 mM NaCl, 0.5% sodium deoxycholate, and 0.2% SDS). The cells were washed again, once in a high salt buffer (0.5M NaCl, 50 mM Tris at pH 7.4) and once in a low salt buffer (10 mM Tris at pH 7.4), to remove the remaining cell lysis buffer. The cells were dissolved in a 2× sample buffer at 95° C. for 5 minutes and centrifuged. Electrophoresis and Western blot analysis were performed with the supernatant according to general methods. Fibronectin monoclonal antibodies and type 1 collagen monoclonal antibodies were used to identify fibronectin and collagen, respectively. For quantitative analysis, fibronectin and collagen bands were visualized by enhanced chemiluminescence (ECL) densitometry, and compared to a control sample. The primary monoclonal antibodies used were Fibronectin (Hybridoma), Collagen I (Quartett), and Cyclin D1 (Dako).

### EXAMPLE 14

### Immunofluorescent Staining

For immunofluorescent staining, coverslips on which cells were inoculated were fixed in 100% methanol and made permeable with 0.2% TritonX-100 in PBS. The cells were reacted with 20% normal goat serum (NGS) diluted in PBS for 1 hour to block nonspecific binding of an antigen. Following this, the cells were reacted overnight at 4° C. with human fibronectin hybridoma culture supernatant (Hybridoma) or α-smooth muscle actin antibodies (Dako), and then with fluorescein-conjugated secondary antibodies for 1 hour at room temperature. The cells were stained with DAPI for 5 minutes to observe the shape of cell nuclei and count the number of cells. The coverslip with the stained cells was mounted in Vectashield (Vector Laboratory). The cells were fluorescently photographed with a fluorescent microscope (BX-FLA, Olympus, Japan).

### EXAMPLE 15

### Immunostaining

For immunostaining, culture plates containing coverslips on which cells were inoculated were fixed in 100% methanol and made permeable with 0.2% TritonX-100 in PBS. Next, the bottoms of the culture plates were removed. The cells were reacted with 20% normal goat serum (NGS) diluted in PBS for 1 hour to block nonspecific binding of an antigen. Following this, the cells were reacted with primary collagen IV antibodies (Dako) at room temperature for 45 minutes, stained by a standard ABC kit (Vector Laboratories), and mounted in Vectashield (Vector Laboratories).

### EXAMPLE 16

### Zymography

Following cell preconditioning with a FX4000T™, activity of MMPs present in cell culture media were analyzed by zymography. Cell culture media were diluted in a sample buffer without mercaptoethanol, and sodium dodecyl sulfate polyacrylamide gel electrophoresis (SDS-PAGE) was carried out using 10% gels containing 0.1% gelatin. After electrophoresis, the gels were renaturated twice, for 30 minutes each time, in 2.5% Triton X-100 at room temperature. Then, the gels were incubated in a 1× developing buffer (50 mM Tris at pH 7.4, 5 mM CaCl₂, and 1M ZnCl₂) at room temperature for 30 minutes, and then incubated with a fresh developing buffer at 37° C. for more than 16 hours. The gels were then stained for 2 hours at room temperature in a staining buffer (10% acetic acid, 10% propanol, and 0.5% Coomassie brilliant blue) and destained in a destaining buffer (10% acetic acid and 10% propanol) until bands appeared. After rinsing with distilled water, the gels were dehydrated in a solution containing 10% glycerol and 12% ethanol.

### EXAMPLE 17

### ELISA for Vascular Endothelial Growth Factor (VEGF)

After preconditioning HUVECs in culture media with the application of strain using a FX-4000T™, variations in the levels of VEGF secretion in the culture media were determined by ELISA using a R&D Qunatikine kit.

Effects of the Invention

1. Cell Yield

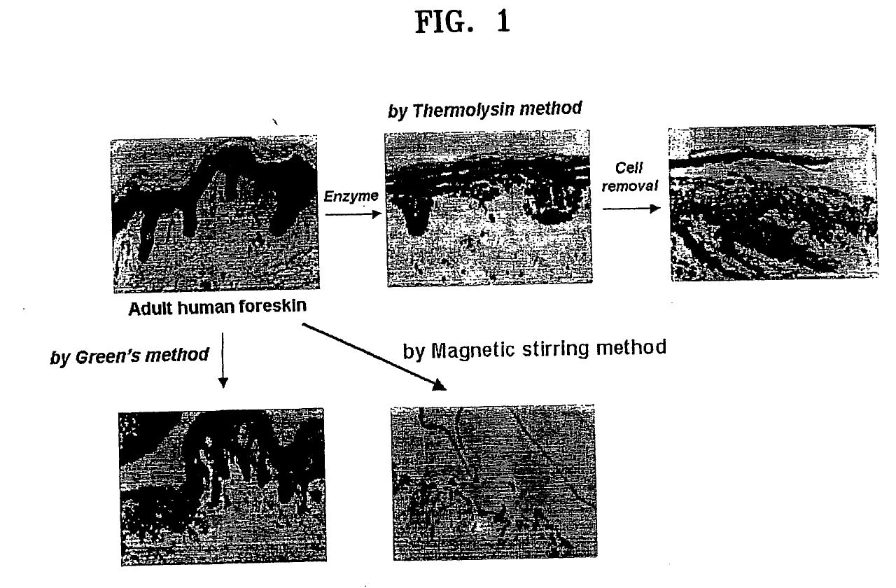

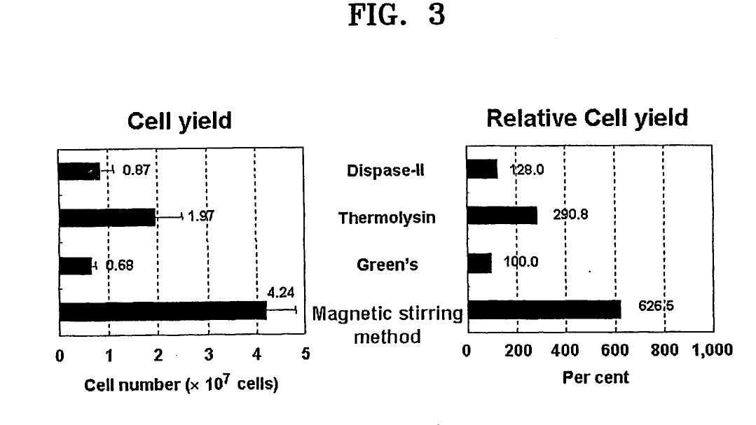

After cells were isolated from tissue according to the four methods, the remaining tissue was fixed and stained with hematoxylin and eosin to determine whether cells remained in the tissue. In tissue from which cells were isolated by the magnetic stirring method, cells rarely existed. In contrast, a large number of stem cells existed in tissue from which cells were separated by the other isolation methods (FIG. 1). This complete isolation of cells from tissue was made possible by the application of magnetic stirring. The effect of magnetic stirring was supported by counting the number of isolated cells (Table 1 and FIGS. 2 and 3). The magnetic stirring method according to the present invention showed about 700% improved cell yield, compared to Green's method.

TABLE 1
Cell Yield - Total Number of Cells per Foreskin Sample (×10⁷)

| Method | Magnetic Stirring | Green's | Thermolysin | Dispase |
|---|---|---|---|---|
| Mean¹ | 4.24 ± 0.57 | 0.68 ± 0.07 | 1.97 ± 0.51 | 0.87 ± 0.30 |
| Range | 2.34˜6.76 | 0.60˜0.88 | 0.36˜3.25 | 0.11˜2.24 |

¹Mean ± SEM

2. Cell Purity

To determine the purity of cells isolated by the different isolation methods, cell cultures were fluorescently stained using Pan-cytokeratin antibodies as a keratinocyte indicator. For the magnetic stirring method, 100% Pan-cytokeratin-positive cells (keratinocytes) were detected. It is evident that cells separated by the magnetic stirring method include pure keratinocytes without fibroblasts (FIG. 8). The same ratio of Pan-cytokeratin-positive cells was detected in cell cultures for the other cell isolation methods. Therefore, the magnetic stirring method provided the same effect as the other isolation methods for cell purity.

3. Colony Forming Efficiency (CFE)

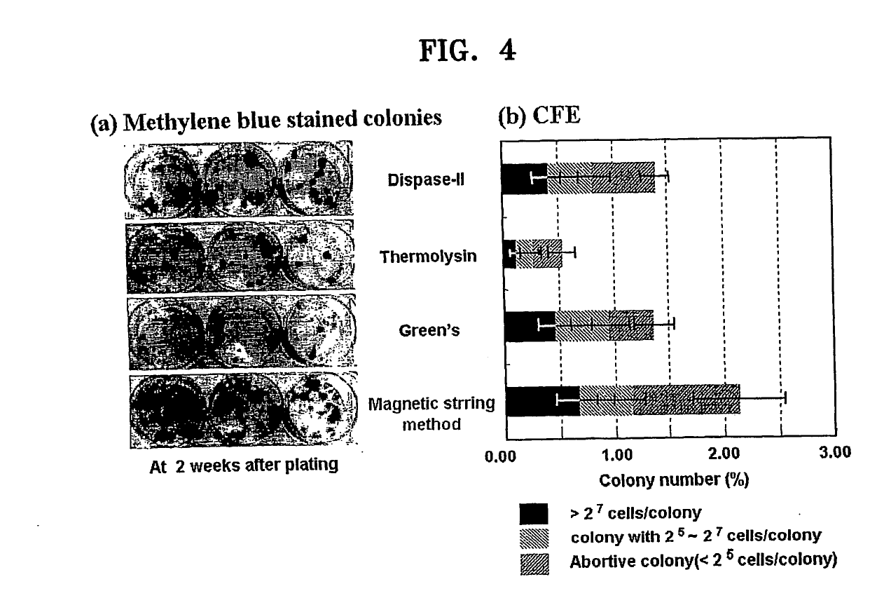

The presence of stem cells can be determined by CFE. Keratinocytes isolated by the magnetic stirring method showed the highest CFE, compared to the other isolation methods (Table 2, FIG. 4). In particular, the CFE for a large colony (including more than 128 cells) was markedly increased (Table 2). These results indicate that the ratio of stem cells is greatly improved in the culture of keratinocytes isolated by the magnetic stirring method.

TABLE 2
CFE (%)¹

| | Magnetic | | | |
|---|---|---|---|---|
| Colony Size | Stirring | Green's | Thermolysin | Dispase |
| <32 | 0.979 ± 0.419 | 0.416 ± 0.177 | 0.265 ± 0.123 | 0.571 ± 0.136 |
| >32 | 1.149 ± 0.319 | 0.947 ± 0.345 | 0.275 ± 0.122 | 0.826 ± 0.298 |
| 32-100 | 0.485 ± 0.122 | 0.488 ± 0.199 | 0.163 ± 0.076 | 0.419 ± 0.169 |
| >100 | 0.672 ± 0.213 | 0.461 ± 0.147 | 0.112 ± 0.048 | 0.407 ± 0.140 |

¹After 2-week incubation following seeding of 10,000 cells on each 6-well plate

Cells isolated by the magnetic stirring method according to the present invention showed greater CFE and cell yield, compared to the other cell isolation methods. Therefore, it is apparent that cell yield and CFE can be improved by physical force generated by magnetic stirring. In conclusion, according to the present invention, the total number of colony forming cells per foreskin sample was improved 9 times more (FIG. 5).

In addition, low intake rate in adult skin grafting caused by the presence of insufficient stem cells in an implanted construct can be compensated for by the present invention.

4. Integrin Expression

As a result of immunostaining, α₂ integrin that is specific to the cells present in the basement membrane (basal cells), is expressed in all keratinocytes isolated by the magnetic stirring method (FIG. 8). This result indicates that in vitro cell expansion is caused by the division of basal keratinocytes.

Flow cytometry with β₁ integrin is a relative measure of the ratio of β₁ integrin-bright cells as a stem cell indicator, in the cultures of skin keratinocytes isolated by the different isolation methods. In the culture of skin keratinocytes isolated by the magnetic stirring method according to the present invention, the distribution of β₁ integrin bright cells is skewed to the right with the highest ratio of stem cells, compared to the cell groups isolated by the other methods (FIG. 6).

5. Involucrin Expression

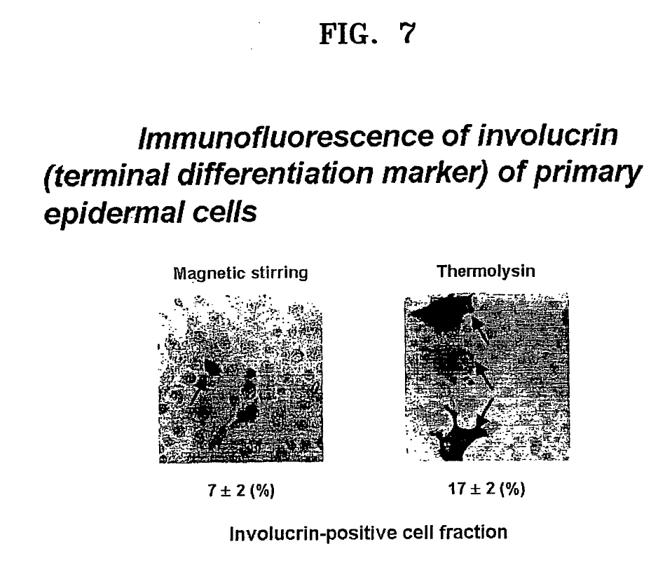

Involucrin as a keratinocyte differentiation marker was expressed at low levels in the culture of keratinocytes: 7% for the magnetic stirring method, 7% for Green's method, 17% for Thermolycin method, and 23% for Dispase method (Table 3, FIG. 7). Cells expressed with involucrin are soon destroyed after undergoing continuous differentiation and aging.

TABLE 3
Percentage of Involucrin Expression

| | Magnetic | | | |
|---|---|---|---|---|
| Method | Stirring | Green's | Thermolysin | Dispase |
| Involucrin + cell | 7 ± 2 | 7 ± 1 | 17 ± 2 | 23 ± 6 |
| (%) | | | | |
| P value | — | — | <0.005 | <0.05 |

6. In Vivo Differentiation of Keratinocytes

Skin keratinocyte and dermal fibroblast cultures implanted into the backs of mice were differentiated into perfect skin consisting of the epidermis, basement membrane, and dermis (FIG. 10). Keratinocytes were positive in human-specific Pan-cytokeratin expression, and dermal fibroblasts were positive in human-specific Vimentin expression. This result indicates that those keratinocytes and dermal fibroblasts were derived from human. In addition, it is apparent that keratinocytes and fibroblasts alive near the wound site of nude mice also migrate together and differentiate into the epidermis and the dermis, respectively. In addition the basement membrane was successfully regenerated between human epidermis and human dermis.

7. In Vitro Differentiation of Keratinocytes

Keratinocytes differentiate into the stratified multilayer of epidermis in a natural state. To investigate the differentiation capability in keratinocytes isolated by the magnetic stirring method according to the present invention, the culture of isolated keratinocytes was directly inoculated in a de-epidermized dermis (DED), fixed, and stained with H&E. As a result, keratinocytes that are positive in Pan-cytokeratin expression, were observed as grown into multiple layers (FIG. 11).

8. Bioartificial Dermis Obtained by Inoculating Fibroblasts in Artificial Dermal Construct

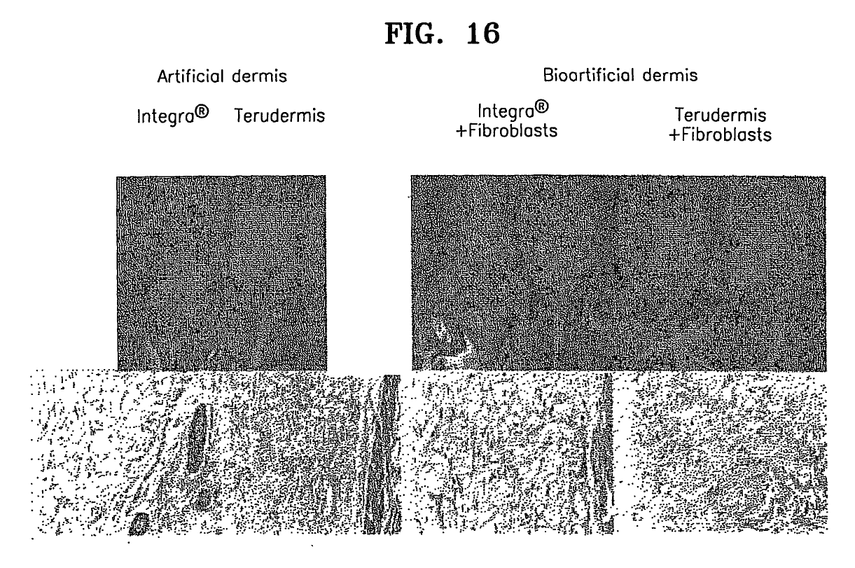

When fibroblasts were inoculated and cultured under dynamic conditions by applying strain, the number of dermal fibroblasts adhering to a Bioartificial skin construct BAS™ was increased, compared to those inoculated and cultured under static conditions (FIG. 16). Scanning electromicroscopic (SEM) photographs of the dermal fibroblasts in BAS™ show that secretion of extracellular matrix components was rich in the attached cells (FIG. 12). This result supports that cells in bioartificial dermis function as in vivo. Unlike dermal fibroblasts inoculated in BAS™ which are concentrated in the surface of the structure, dermal fibroblasts inoculated in a DED are found deep within the structure and have comparatively uniform distribution with almost the same confluency as in real intact dermis. Dermal fibroblasts inoculated in artificial dermal constructs, Integra® and Terumdermis, showed uniform distribution and similar confluency to that in DED.

9. Structure of Bioartificial Dermis and Artificial Dermal Construct Implanted into Nude Mouse

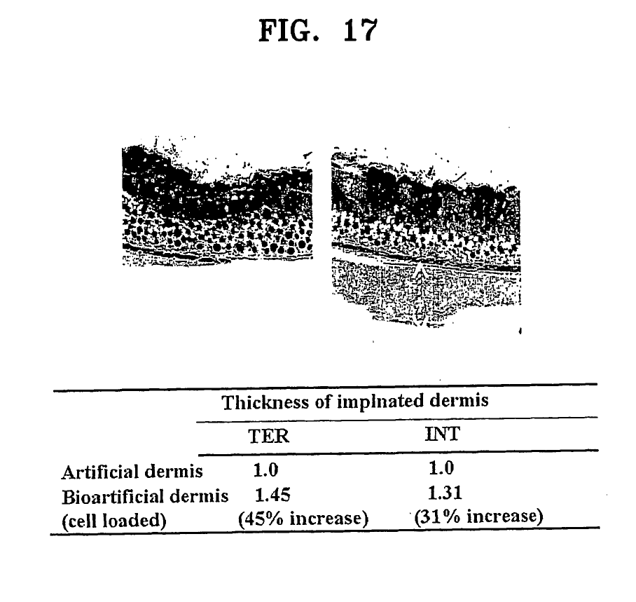

Bioartificial dermis (FIG. 14) obtained by incubating fibroblasts in Integra® and Terumdermis for 14 days, and commercially available Integra® and Terumdermis (FIG. 15) were implanted into nude mice and stained with H&E (FIGS. 15 and 16). No sign of inflammation was observed in the implant sites or neighboring tissue. The implant sites were fused well into neighboring tissue and maintained initial sizes (FIG. 16). Incorporation of dermal fibroblasts and blood vessels was observed over the implant sites with similar fibroblast confluency to intact murine dermis (FIG. 16). Variations in height of the implant sites were too small to be measured with a calibre, so the heights of the implant sites were measured based upon the photographs of tissue staining (FIG. 17). Volume reductions at implant sites were observed for both Integra® and Terumdermis. The reason for this is considered to be collagen contraction and implant dissolution. The level of volume reduction in implants was smaller in the bioartificial dermis inoculated with viable cells than in artificial dermal constructs, particularly smaller in Integra® than Terumdermis (FIG. 17).

Bioartificial skin or dermis according to the present invention can be applied to larger wound sites usually caused by burns, or tissue damage caused by diabetes where cells near the wound site cannot easily migrate. Also, bioartificial skin or dermis according to the present invention can readily be used to generate tissue depressed by plastic surgery.

10. Increase in the Number of Cells by Application of Strain

When dermal fibroblasts were preconditioned at 37° C. for 2 days with the application of strain using a FX-4000T™ at a frequency of 1.0 Hz at 10% maximum strain, total protein content was increased about 4.8 times, compared to a control group, increased about 2.1 times with the addition of platelet-derived growth factor (PDGF-BB), increased about 1.3 times with the addition of insulin-like growth factor (IGF-I), and increased about 1.3 times with the addition of both PDGF-BB and IGF-I (Table 4).

TABLE 4
Total Protein Content (mg/mL)

| Group | No Strain Applied | Strain Applied | Factor of Increase |
|---|---|---|---|
| Control | 1.363 | 6.485 | 4.8 |
| PDGF-BB | 3.101 | 6.393 | 2.1 |
| IGF-l | 4.656 | 6.027 | 1.3 |
| PDGF-BB + | 7.308 | 9.137 | 1.3 |
| IGF-I | | | |

The number of cells visualized by phase contrast microscopy showed almost the same pattern as the increase in protein content (FIG. 19). The number of cells was markedly increased in the group to which strain was applied, compared to the group to which strain was not applied (A and B of FIG. 19). The increase in the number of cells by the application of strain was greater than in the groups treated with PDGF-BB (50 ng/mL), IGF-I (10 ng/mL), and PDFG-BB+IGF-I without the application of strain (B, C, E, and G of FIG. 9, and A, C, E & G of FIG. 9) When PDGF-BB (50 ng/mL), IGF-I (10 ng/mL), and PDGF-BB+IGB-I were added simultaneously with the application of strain, there were similar increases in the number of cells to the groups to which strain was applied without the addition of growth factor (B, D, F, and H of FIG. 19).

The increase in the number of cells caused by the application of strain was smaller in adult dermal fibroblasts than in newborn dermal fibroblasts. This is because newborn dermal fibroblasts is more sensitive to strain than adult dermal fibroblasts.

11. Mitogenic Protein Expression by Application of Strain

When dermal fibroblasts were preconditioned at 37° C. for 2 days with the application of strain using a FX-4000T™ at a frequency of 1.0 Hz at 10% maximum strain, the level of Cyclin-D1 expression was increased about 8 times compared to a control group. Compared with the groups to which growth factors were added without the application of strain, the groups to which both growth factor and strain were applied showed increased expression of Cyclin-D1 of 26-29 times (FIG. 20, Table 5).

TABLE 5
Relative Comparison of Cyclin-D1 Expression

| Group | No Strain Applied | Strain Applied | Factor of Increase |
|---|---|---|---|
| Control | 1.0 | 9.2 | 9 |
| PDGF-BB | 0.3 | 8.7 | 29 |
| IGF-l | 0.1 | 7.0 | 70 |
| PDGF-BB + | 0.2 | 5.2 | 26 |
| IGF-l | | | |

12. Increase in Secretion of Extracellular Matrix Component (Fibronectin, Collagen) by Application of Strain

When newborn dermal fibroblasts were preconditioned at 37° C. for 2 days with the application of strain using a FX4000T™ at a frequency of 1.0 Hz at 10% maximum strain, the level of secretion of fibronectin in cell culture media was increased about 282 times compared to a control group. This was an increase of a maximum of 94 times and a minimum of 2.8 times in comparison to the groups to which PDGF-BB (increased 3 times more the control group), IGF-I (increased 22 times more the control group), and both PDGF-BB and IGF-I (increased 108 times more the control group), were added (A of FIG. 21). The level of secretion of fibronectin was increased 282 times with the application of only strain. Secretion of fibronectin was increased about 3.2 times more for the groups treated with PDGF-BB and IGF-I simultaneously with the application of strain. However, secretion of type I collagen was not affected by the application of strain (A of FIG. 21).

For adult dermal fibroblasts, although they are less sensitive to strain than newborn dermal fibroblasts are, fibronectin secretion was increased by the application of strain by about 2.6 times as in the group treated with only PDGF-BB or IGF-I (B of FIG. 21).

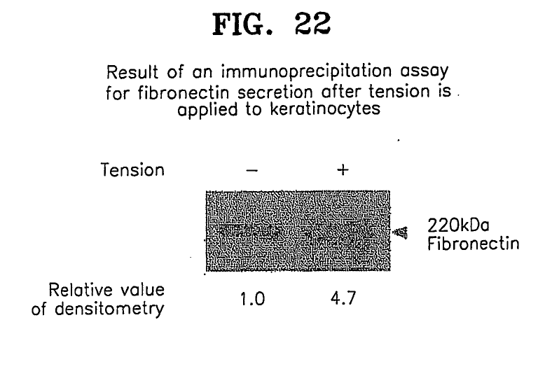

When skin keratinocytes were preconditioned at 37° C. for 2 days with the application of pulsatile strain using a FX-4000T™ at a frequency of 0.5 Hz at 20% maximum strain, the level of secretion of fibronectin in cell culture media was increased about 4.7 times compared to a control group (FIG. 22).

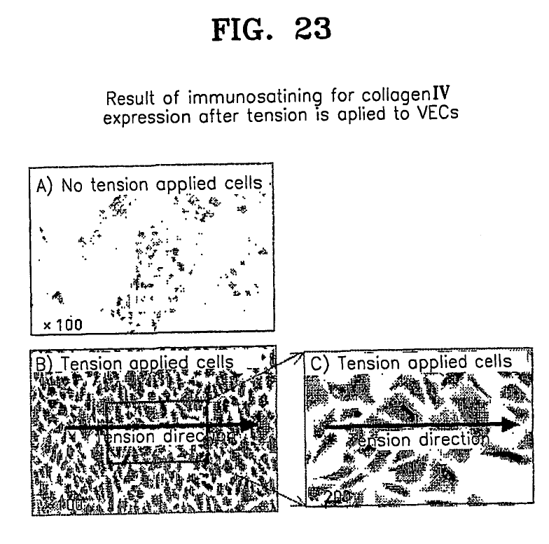

When vascular endothelial cells were preconditioned for 2 days with the application of strain using a FX4000T™ at a frequency of 1.0 Hz at 10% maximum strain, the expression of collagen IV was markedly increased (A and B of FIG. 23). In particular, as a result of high-power microscopy, a complex filamentous web of collagen IV was observed in the base of vascular endothelial cells (C of FIG. 23).

Collagen IV is essential for vascular epithelial cells to form blood vessels. Therefore, the increase in synthesis of collagen IV and distribution of collagen IV in the base of the cells are expected to stimulate generation of blood vessels.

13. Verification of the Preconditioning Effect Caused by the Application of Strain Lasting after Sub-Culturing

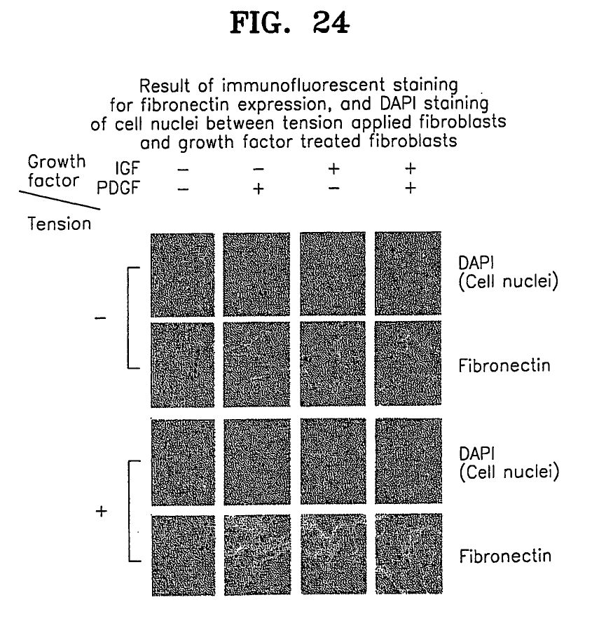

When adult dermal fibroblasts preconditioned at 37° C. for 2 days with the application of strain using a FX-4000T™ at a frequency of 1.0 Hz at 10% maximum strain were subjected to trypsinization and sub-culturing, the level of fibronectin expression increased after 4 days (FIG. 24) and 7 days.

14. Verification of Increase in the Number of Pure Fibroblasts by the Application of Strain

As a result of immunofluorescent staining after treatment with trypsin and sub-culturing, on adult fibroblasts preconditioned at 37° C. for 2 days with the application of strain using a FX-4000T™ at a frequency of 1.0 Hz at 10% maximum strain, the cells showed negative expression of α-smooth muscle actin acting as a myofibroblast indicator (FIG. 25). This result supports that the features of fibroblasts are maintained after the application of strain. However, the groups treated with growth factors showed a sharp increase in cells that are positive in α-smooth muscle actin expression (FIG. 25), which means that a considerable number of cells were differentiated into myofibroblasts after the treatment of growth factors. In wound healing periods, myofibroblasts appear as a passing phenomenon. However, if myofibroblasts exist for a while during the wound healing period, it is highly likely that scar is formed, and fibroblasts provide more crucial functions than do myofibroblats in wound curing periods. Therefore, the groups to which strain was applied are expected to have excellent wound healing effect, compared to the groups treated with growth factors.

15. Increase in Activity of MMPs by Application of Strain

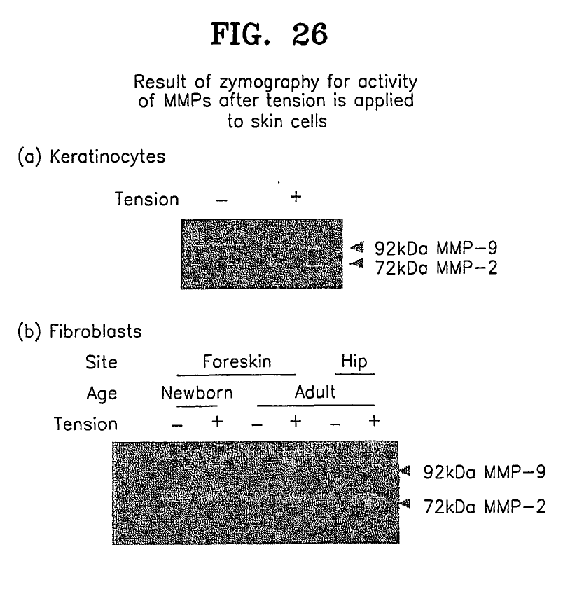

When skin fibroblasts were preconditioned at 37° C. for 2 days with the application of pulsatile strain using a FX4000T™ at a frequency of 1.0 Hz at 10% maximum strain, the activities of matrix metalloproteinase (MMP)-2 and MMP-9 in cell culture media were improved, compared to a control group (A of FIG. 26).

When skin keratinocytes were preconditioned at 37° C. for 2 days with the application of pulsatile strain using a FX4000T™ at a frequency of 0.5 Hz at 20% maximum strain, the activity of MMP-9 in cell culture media were improved with no significant change in the activity of MMP-2, compared to a control group (B of FIG. 26).

16. Verification of Therapeutic Applicability of Allogeneic Fibroblasts by Measuring HLA-ABC Expression Reduction Caused by Fibroblast Sub-Culturing

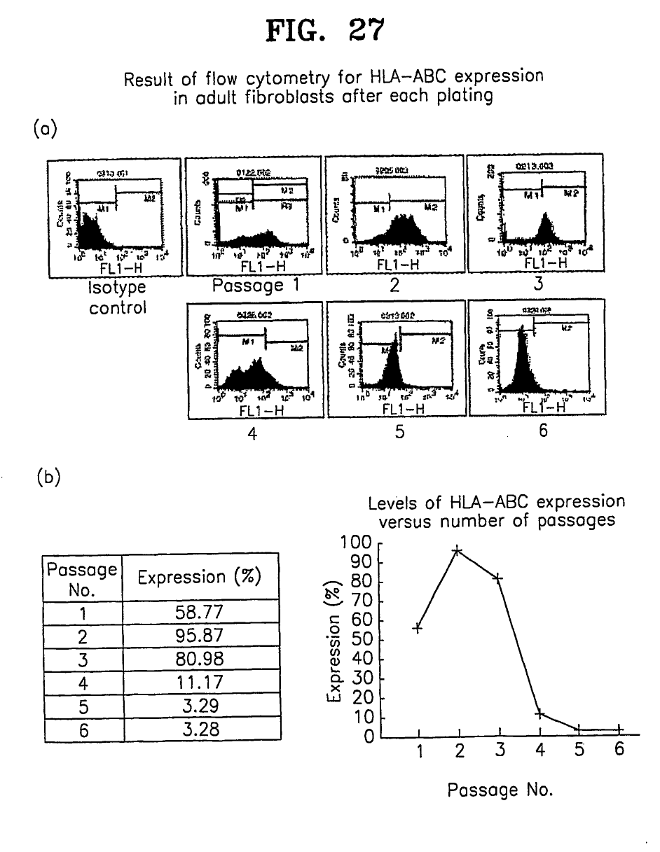

HLA-ABC expression in dermal fibroblasts was about 56.77% in the first passage and increased to 85.87% in the second passage. HLA-ABC expression in dermal fibroblasts decreased to 60.96% in the third passage and sharply decreased to 11.17% in the fourth passage. HLA-ABC was rarely expressed in the fifth passage of the dermal fibroblasts at 3.29% and was almost the same in the next passage. Thus, it is apparent that HLA-ABC expression mostly disappears in the fifth passage of dermal fibroblasts (FIG. 27). From this result, it is evident that biological allogeneic dermal fibroblasts can be used as a therapeutic cell resource after being undergone four or more passages without histo-incompatibility.

17. Increase in Vascular Endothelial Growth Factor (VEGF) Secretion in Vascular Endothelial Cells by Application of Strain

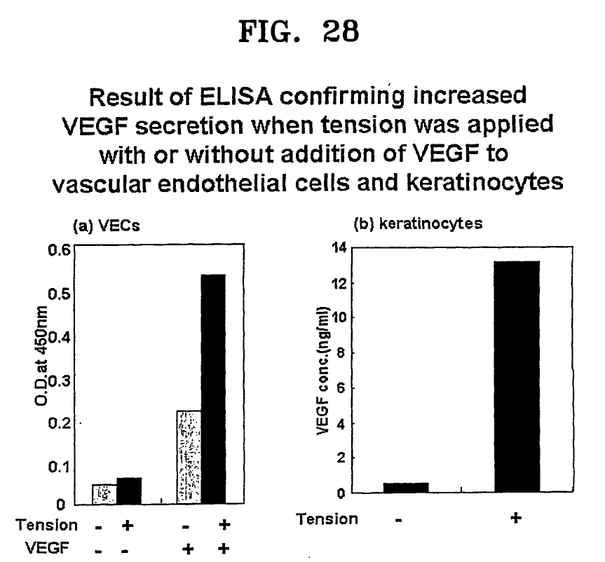

When vascular endothelial cells (VECs) were preconditioned for 2 days with the application of strain using a FX4000T™ at a frequency of 1.0 Hz at 15% maximum strain, the level of VEGF secretion was increased about 30%, and increased about 200% with the addition of 10 ng/mL VEGF (FIG. 28). When strain was applied to keratinocytes, the level of VEGF secretion increased about 2,400% (FIG. 28). Therefore, the application of strain stimulated the secretion of VEGF in both VECs and keratinocytes.

As described above, according to the present invention, cell viability and mitogenetic capability after implantation can be improved by preconditioning cell cultures against stress and physical stimuli which the cells would undergo after implantation, by the application of strain during incubation of cell cultures to be implanted. As a result, the time required for cell propagation can be reduced with increased synthesis and secretion of fibronectin, which is known to be essential for wound healing, and with increased activity of matrix metalloproteinases (MMPs), thereby facilitating wound recovery. In addition, synthesis of collagen IV is also increased so that formation of blood vessels is facilitated. These advantages of cell preconditioning improve the capability of integration into host tissue and ensure successful skin grafting.

1. A method of isolating epithelial cells by treating skin tissue or internal organ tissue with trypsin or trypsin and EDTA simultaneously with magnetic stirring.

2. The method of claim 1, wherein the skin tissue is obtained from the foreskin, axilla, hip, breast, scalp, cornea, pubes, abdomen or marsupium.

3. The method of claim 1, wherein the internal organ tissue is obtained from the oral cavity mucosa, esophagus mucosa, gastric mucosa, intestinal mucosa, nasal cavity mucosa, gorge, kidney, urethra, uterus mucosa, bladder, or vagina.

4. The method of claim 1, wherein, when the skin tissue or internal organ tissue is treated with only trypsin, the trypsin is added in an amount of 0.025-0.25%, and when the skin tissue or internal organ tissue is treated with trypsin and EDTA, the trypsin is added in an amount of 0.025%-0.25%, and the EDTA is added in an amount of 0.005-0.02%.

5. The method of claim 1, wherein the magnetic stirring is carried out at 60-700 rpm for 10 minutes to 4 hours.

6. A method of preparing a bioartificial skin by inoculating the epithelial cells isolated by the method of any of claims 1 through 5 in an artificial dermal construct or de-epidermized dermis (DED) exclusively or along with fibroblasts.

7. The method of claim 6, wherein the epithelial cells are inoculated in a bioartificial dermis prepared by inoculating fibrobroblasts in an artificial dermal construct or de-epidermized dermis (DED).

8. The method of claim 6 or 7, wherein the epithelial cells are inoculated together with melanocytes.

9. The method of claim 6 or 7, wherein the epithelial cells are inoculated together with hair follicle cells or dermal sheath.

10. The method of claim 6 or 7, wherein the epithelial cells are inoculated together with vascular endothelial cells.

11. A method of healing damaged skin or internal organ by implanting the epithelial cells isolated by the method of any of claims 1 through 5 in a damaged skin tissue or internal organ tissue exclusively or along with fibroblasts.

12. A method of healing damaged skin or internal organ by implanting the bioartificial skin prepared by the method of any of claims through 10 in a damaged skin tissue or internal organ tissue.

13. The method of claim 11 or 12, wherein the skin tissue is a skin site damaged by burns, traumatic injury, or ulcer, or a skin site which needs dermatoplastic surgery, tissue expansion and augmentation, or cornea implantation.

14. The method of claim 11 or 12, wherein the damaged internal organ tissue is a damaged tissue site which needs restitution or regeneration after having undergone incision or radiotherapy to cure cancer or for other purposes.

15. A method of preconditioning cells isolated from the body in cultures with the application of physical stimuli.

16. The method of claim 15, wherein the cells are fibroblasts.

17. The method of claim 15, wherein the cells are vascular endothelial cells.

18. The method of claim 15, wherein the cells are keratinocytes.

19. A method of preparing a bioartificial dermis by inoculating the cells cultured by the method of claim 15 in an artificial or native dermal construct.

20. A method of preparing a bioartificial dermis with the application of physical stimuli after inoculating cells in an artificial or native dermal construct.

21. The method of claim 19 or 20, wherein the native dermal construct is at least one selected from the group consisting of de-epidermized dermis (DED), collagen solution, fibrin solution, gelated collagen, and gelated fibrin, and the artificial dermal construct is at least one selected from the group consisting of neutralized chitosan sponge, a mixed sponge of neutralized chitosan and collagen, Integra®, Alloderm, Terudermis, and Beschitin W.

22. The method of claim 19 or 20, wherein the cells include fibroblasts and/or vascular endothelial cells.

23. The method of claim 19 or 20, wherein fibronectin and/or glycoseaminoglycan are added to the artificial or native dermal construct.

24. A method of preparing a bioartificial skin by inoculating keratinocytes preconditioned by the method of claim 18 in a dermal construct exclusively or along with melanocytes, dermal sheath, or hair follicle cells.

25. A method of preparing a bioartificial skin by the application of physical stimuli after inoculating keratinocytes exclusively or along with melanocytes in a dermal construct.

26. The method of claim 24 or 25, wherein the dermal construct includes artificial and native dermal constructs, bioartificial dermal constructs, and the bioartificial dermis prepared by the method of claim 19 or 20.

27. The method of any of claims 15, 20, and 25, wherein the physical stimuli include pulsatile or continuous strain applied at a frequency of 0.1-3.0 Hz at 0.01-40% maximum strain.

28. The method of claim 26, wherein the native dermal construct is at least one selected from the group consisting of de-epidermized dermis (DED), collagen solution, fibrin solution, gelated collagen, and gelated fibrin, and the artificial dermal construct is at least one selected from the group consisting of neutralized chitosan sponge, a mixed sponge of neutralized chitosan and collagen, Integra®, Alloderm, Terudermis, and Beschitin W.

29. A method of healing a damaged tissue by implanting the bioartificial dermis prepared by the method of claim 19 or 20 or the bioartificial skin prepared by the method of claim 24 or 25 in a damaged skin tissue or internal organ tissue.

30. A method of curing a damaged tissue by directly implanting the fibroablasts preconditioned by the method of claim 16, the vascular endothelial cells preconditioned by the method of claim 17, the keratinocytes preconditioned by the method of claim 18 separately or together in a damaged skin tissue or internal organ tissue.
